(19) United States
(12) Patent Application Publication
(10) Pub. No.: US 2017/0245800 A1
(43) Pub. Date: Aug. 31, 2017

(54) BIOLOGICAL-INFORMATION ANALYZING DEVICE, BIOLOGICAL-INFORMATION ANALYZING SYSTEM, AND BIOLOGICAL-INFORMATION ANALYZING METHOD

(71) Applicant: SEIKO EPSON CORPORATION, Shinjuku-ku, Tokyo (JP)

(72) Inventors: Ichiro AOSHIMA, Matsumoto-shi (JP); Yusuke TAKAHASHI, Matsumoto-shi (JP); Atsushi NARUSAWA, Chino-shi (JP); Hidekazu MAEZAWA, Shiojiri-shi (JP)

(73) Assignee: SEIKO EPSON CORPORATION, Tokyo (JP)

(21) Appl. No.: 15/527,263

(22) PCT Filed: Nov. 16, 2015
(86) PCT No.: PCT/JP2015/005687

§ 371 Date: May 16, 2017

(30) Foreign Application Priority Data
Dec. 1, 2014 (JP) 2014-243524

(51) Int. Cl. A61B 5/00; A61B 5/11; A61B 5/0205; A61B 5/029

(52) U.S. Cl. CPC A61B 5/72; A61B 5/029; A61B 5/11; A61B 5/742; A61B 5/0205; A61B 2503/10; A61B 5/0245

(57) ABSTRACT

To provide a biological-information analyzing device, a biological-information analyzing system, and a biological-information analyzing method that can appropriately measure biological information even in a user having an athlete's heart. A biological-information analyzing device includes a biological-information detecting section configured to detect biological information of a user, a user determining section configured to determine an exercise capability of the user, an information setting section configured to set, when it is determined that the exercise capability of the user satisfies a predetermined condition, analysis information corresponding to the exercise capability of the user, and an analyzing section configured to analyze the biological information on the basis of the set analysis information.

### TECHNICAL FIELD

The present invention relates to abiological-information analyzing device, a biological-information analyzing system, and a biological-information analyzing method.

### BACKGROUND ART

There has been known a pulsimeter worn on a user to measure a pulse rate serving as biological information of the user. The pulsimeter includes a pulse wave sensor, which uses light and ultrasound, and calculates a pulse rate on the basis of a change in a blood flow amount of the user detected by the pulse wave sensor.

A pulse wave signal detected by the pulsimeter is a signal in which a pulsation component signal and a body motion component signal of the user are superimposed. Therefore, when a body motion of the user is intense, it is likely that a ratio of the body motion component signal is high with respect to the pulsation component signal and a pulse rate cannot be appropriately calculated.

On the other hand, there has been proposed a pulsimeter including, in addition to the pulse wave sensor, a body motion sensor that detects body motion information of a user (see, for example, PTL 1).

The pulsimeter described in PTL 1 calculates exercise intensity of the user on the basis of the body motion information detected by the body motion sensor (an acceleration sensor) and estimates a pulse rate corresponding to the calculated exercise intensity on the basis of a predetermined relation between exercise intensity and a pulse rate. When calculation of a pulse rate based on a detection result by the pulse wave sensor cannot be correctly performed, the pulsimeter presents the estimated pulse rate to the user.

### CITATION LIST

### Patent Literature

PTL 1: JP-A-2012-232010

### SUMMARY OF INVENTION

### Technical Problem

Incidentally, beside a person not having exercise habits and a person having exercise habits, a person having a high cardiopulmonary function called athlete's heart could be the user of the pulsimeter. The athlete's heart indicates a heart itself enlarged to be larger than usual as a result of the body of the user being adapted to an improved athletic capability or an excellent cardiopulmonary function obtained by having such a heart.

Examples of characteristics of the person having the athlete's heart include low pulse rates during a rest and during exercise. This occurs because, as a result of the development of the muscle of the heart, a larger amount of blood can be delivered by one pulsation and sufficient blood and oxygen can be carried to the entire body even by a small number of pulsations.

Note that a condition for determining that a person is a person have an athlete's heart (hereinafter referred to as determination condition) is that, for example, a cardiac output per one stroke is 150 ml or more, more desirably, 200 ml or more or a resting pulse rate is 50 or less, more desirably, 40 or less. The cardiac output and the resting pulse rate can be extracted by analyzing a pulse wave or an electrocardiogram. The athlete's heart can be rephrased as exercise capability or cardiopulmonary capability. The determination condition can be set as, for example, time of a long-distance race.

In the person having the athlete's heart, a change in a pulse rate involved in exercise is different from the change of the pulse rate of an ordinary person. Therefore, it is likely that the pulsimeter described in PTL 1 cannot appropriately calculate and measure a pulse rate of a user having an athlete's heart.

Because of such a problem, there has been a demand for a configuration capable of appropriately detecting a pulse rate of even the user having the athlete's heart.

An object of the invention is to solve at least a part of the problems. One of objects of the invention is to provide a biological-information analyzing device, a biological-information analyzing system, and a biological-information analyzing method that can analyze biological information according to a user.

### Solution to Problem

A biological-information analyzing device according to a first aspect of the invention includes: a biological-information detecting section configured to detect biological information of a user; a user determining section configured to determine an exercise capability of the user; an information setting section configured to set, when the user determining section determines that the exercise capability of the user satisfies a predetermined condition, analysis information corresponding to the exercise capability of the user; and an analyzing section configured to analyze the biological information on the basis of the analysis information set by the information setting section.

According to the first aspect, when it is determined that the exercise capability of the user of the biological-information analyzing device satisfies the predetermined condition, the biological information of the user is analyzed on the basis of the analysis information corresponding to the exercise capability of the user. Consequently, it is possible to execute analysis processing of the biological information corresponding to the exercise capability of the user. Therefore, it is possible to analyze and measure the biological information appropriately for the user.

In the first aspect, it is preferable that the exercise capability is a capability concerning a cardiopulmonary function.

Note that stamina can be illustrated as the capability concerning the cardiopulmonary function.

According to the first aspect, when it is determined that the capability concerning the cardiopulmonary function of the user satisfies the predetermined condition as the exercise capability, the biological information is analyzed on the basis of analysis information corresponding to the capability. Consequently, it is possible to more appropriately carry out the analysis of the biological information related to the cardiopulmonary function of the user. Therefore, it is possible to more appropriately analyze and measure the biological information.

In the first aspect, it is preferable that the user determining section determines the capability concerning the cardiopulmonary function on the basis of at least one of a resting pulse rate and a cardiac output.

In a person having an excellent cardiopulmonary function like the person having the athlete's heart, the resting pulse rate is low and the cardiac output is large compared with an ordinary person (e.g., a person not having exercise habits). Therefore, by determining the capability concerning the cardiopulmonary function (the exercise capability) of the user on the basis of at least one of the resting pulse rate and the cardiac output, the user determining section can appropriately determine the capability.

In the first aspect, it is preferable that the biological-information analyzing device further includes an operation section configured to receive input operation by the user, and the user determining section determines the exercise capability of the user on the basis of content of the input operation.

According to the first aspect, the exercise capability of the user is determined on the basis of the content of the input operation by the user. Consequently, for example, when input operation indicating whether the exercise capability of the user is high is performed by the user or when operation for setting analysis information used for the analysis of the biological information is performed by the user, it is possible to determine the exercise capability of the user on the basis of content of the input operation. Therefore, compared with when the determination processing is executed by, for example, analyzing the biological information, it is possible to simply execute the determination processing by the user determining section.

In the first aspect, it is preferable that the biological-information analyzing device further includes a display section configured to display a screen to which user information concerning the user is input, and the user determining section determines the exercise capability of the user on the basis of the input user information.

Note that, as the user information, information indicating the exercise capability of the user can be illustrated. More specifically, time of a long-distance race and a resting pulse rate of the user can be illustrated.

According to the first aspect, the determination processing by the user determining section is executed on the basis of the user information. Therefore, it is possible to more simply and appropriately execute the determination processing.

In the first aspect, it is preferable that the biological-information analyzing device further includes a body-motion-information detecting section configured to detect body motion information of the user, and the user determining section determines the exercise capability of the user on the basis of an exercise state of the user based on the detected body motion information and biological information of the user in the exercise state.

According to the first aspect, the determination processing is executed on the basis of the exercise state of the user and the biological information in the exercise state. Consequently, for example, by comparing biological information of the user actually detected in the exercise state based on the body motion information and an indicator of biological information for each of exercise capabilities that could be detected in the exercise state, it is possible to relatively simply and appropriately determine the exercise capability of the user.

In the first aspect, it is preferable that the biological information includes at least a pulse wave, and the analyzing section calculates a pulse rate of the user on the basis of the pulse wave.

In a person having an excellent exercise capability and a person not having an excellent exercise capability, as explained above, an upper limit value of a pulse rate during exercise and a pulse rate during a rest are different.

On the other hand, according to the first aspect, the analyzing section calculates the pulse rate on the basis of the detected pulse wave. Therefore, even when the user is a person having a high exercise capability, for example, when the user is a person having an athlete's heart, it is possible to appropriately calculate the pulse rate of the user.

In the first aspect, it is preferable that the biological-information analyzing device further includes a body-motion-information detecting section configured to detect body motion information of the user, the analysis information is a table in which an exercise state of the user based on the body motion information detected by the body-motion-information detecting section and a pulse rate are associated, and the analyzing section calculates the pulse rate on the basis of the table.

According to the first aspect, the analyzing section calculates the pulse rate on the basis of the table in which the exercise state based on the body motion information and the pulse rate are associated. Consequently, for example, when a body motion of the user during exercise is intense and biological information cannot be appropriately detected, it is possible to estimate a pulse rate of the user. Therefore, it is possible to present a pulse rate corresponding to the exercise state to the user.

In the first aspect, it is preferable that the biological-information analyzing device further includes a pace calculating section configured to calculate a pace of the user on the basis of the body motion information, the table is a table in which a pace and a pulse rate are associated, and the analyzing section acquires, from the table, a pulse rate corresponding to the pace of the user calculated by the pace calculating section and calculates the pulse rate of the user on the basis of the pulse rate and a pulse rate based on the biological information.

According to the first aspect, the pulse rate corresponding to the calculated pace of the user is calculated on the basis of the table selected by the user. Consequently, as explained above, even when the body motion of the user is intense, it is possible to simply estimate a pulse rate corresponding to a pace of exercise being carried out. Therefore, it is possible to more simply present a pulse rate corresponding to an exercise state to the user.

A biological-information analyzing system according to a second aspect of the invention is a biological-information analyzing system that analyzes biological information of a user. The biological-information analyzing system determines an exercise capability of the user, sets, when determining that the exercise capability of the user satisfies a predetermined condition, analysis information corresponding to the exercise capability of the user, and analyzes the biological information on the basis of the set analysis information.

According to the second aspect, it is possible to achieve an effect same as the effect of the biological-information analyzing device according to the first aspect.

A biological-information analyzing method according to a third aspect of the invention is a biological-information analyzing method for analyzing biological information of a user. The biological-information analyzing method includes: determining an exercise capability of the user; setting, when determining that the exercise capability of the user satisfies a predetermined condition, analysis information corresponding to the exercise capability of the user; and analyzing the biological information on the basis of the set analysis information.

According to the third aspect, by carrying out the biological-information analyzing method, it is possible to achieve an effect same as the effect of the biological-information analyzing device according to the first aspect.

A biological-information analyzing system according to a fourth aspect of the invention includes: a detecting device configured to detect biological information of a user; and an analyzing device configured to analyze the biological information detected by the detecting device. The detecting device includes: a biological-information detecting section configured to detect the biological information; and an information transmitting section configured to transmit the biological information detected by the biological-information detecting section. The analyzing device includes: a user determining section configured to determine whether the user is a person having an athlete's heart; an information setting section configured to set, when the user determining section determines that the user is the person having the athlete's heart, analysis information for the athlete's heart in analysis information used for the analysis of the biological information; and an analyzing section configured to analyze, on the basis of the analysis information for the athlete's heart set by the information setting section, the biological information received from the detecting device.

Note that the biological-information analyzing system may be configured by one device in which the detecting device and the analyzing device are integrated or may include the detecting device and the analyzing device independent from each other.

According to the fourth aspect, it is possible to achieve an effect same as the effect of the biological-information analyzing device according to the first aspect.

That is, when the user determining section determines that the user is the person having the athlete's heart, the biological information detected by the biological-information detecting section of the detecting device is analyzed on the basis of the analysis information for the athlete's heart set in the analysis information used for the analysis of the biological information.

Consequently, when the user is the person having the athlete's heart, the analyzing section can analyze the detected biological information on the basis of the analysis information for the athlete's heart. Therefore, it is possible to appropriately analyze and measure the biological information.

A biological-information analyzing method according to a fifth aspect of the invention is a biological-information analyzing method for analyzing biological information of a user. The biological-information analyzing method includes: determining whether the user is a person having an athlete's heart; setting, when it is determined that the user is the person having the athlete's heart, analysis information for the athlete's heart in analysis information used for the analysis of the biological information; and analyzing the biological information on the basis of the set analysis information for the athlete's heart.

According to the fifth aspect, by carrying out the biological-information analyzing method, it is possible to achieve an effect same as the effect of the biological-information analyzing system according to the fourth aspect.

### BRIEF DESCRIPTION OF THE DRAWINGS

FIG. 1 is a block diagram showing the configuration of a biological-information measuring device according to a first embodiment of the invention.

FIG. 2 is a block diagram showing the configuration of a control section in the first embodiment.

FIG. 3 is a diagram showing an example of a setting screen in the first embodiment.

FIG. 4 is a block diagram showing the configuration of a table selecting section in the first embodiment.

FIG. 5 is a flowchart showing first selection processing in the first embodiment.

FIG. 6 is a flowchart showing second selection processing in the first embodiment.

FIG. 7 is a block diagram showing the configuration of an analyzing section in the first embodiment.

FIG. 8 is a flowchart showing pulse rate measurement processing in the first embodiment.

FIG. 9 is a flowchart showing pulse rate calculation processing in the first embodiment.

FIG. 10 is a block diagram showing the configuration of a detecting device configuring a biological-information analyzing system according to a second embodiment of the invention.

FIG. 11 is a block diagram showing the configuration of an analyzing device configuring the biological-information analyzing system in the second embodiment.

FIG. 12 is a block diagram showing the configuration of a control section of the analyzing device in the second embodiment.

### DESCRIPTION OF EMBODIMENTS

### First Embodiment

A first embodiment of the invention is explained below with reference to the drawings.

### [Schematic Configuration of a Biological-Information Measuring Device]

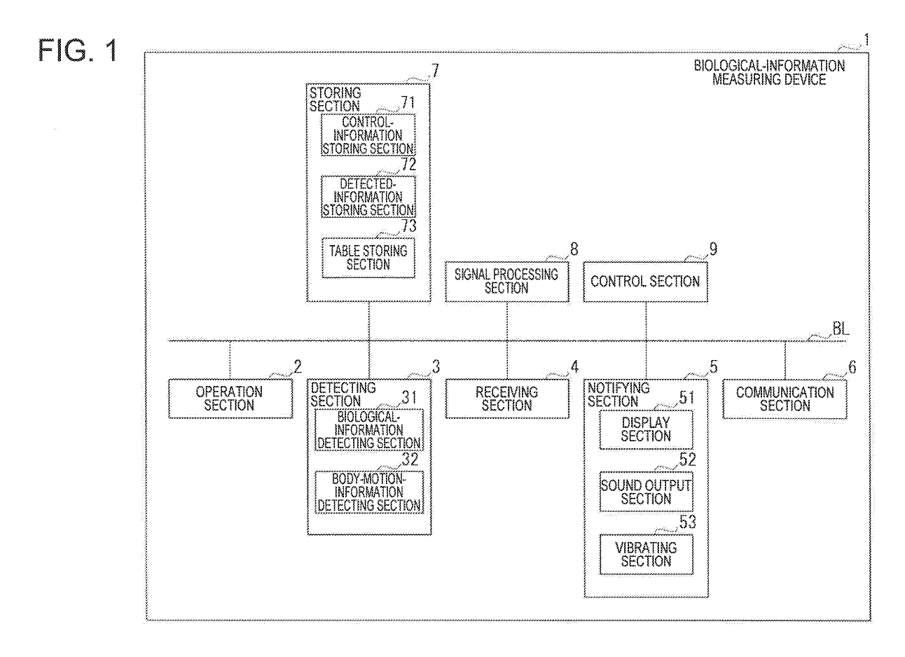

FIG. 1 is a block diagram showing the configuration of a biological-information measuring device 1 according to this embodiment.

The biological-information measuring device (hereinafter sometimes abbreviated as measuring device) 1 according to this embodiment is an wearable device that measures biological information of a user wearing the measuring device 1 in a wearing part such as a wrist and is equivalent to a biological-information analyzing device. The measuring device 1 transmits detected biological information and body motion information to an information processing device (not shown in the figure) that communicates with the measuring device 1.

As explained in detail below, the measuring device 1 has stored therein a plurality of tables serving as analysis information in which a state of exercise to be carried out and an estimated value of a pulse rate at the time when the exercise is carried out are associated. Among the tables, a table corresponding to an exercise capability of the user is selected and an estimated pulse rate is acquired. Besides, in calculation processing for a pulse rate, processing different from processing for an ordinary user is executed. Further, the selected table is learned and updated according to an exercise state of the user. That is, the measuring device 1 is also a pulse-rate measuring device that measures a pulse rate of the user.

The measuring device 1 includes, as shown in FIG. 1, an operation section 2, a detecting section 3, a receiving section 4, a notifying section 5, a communication section 6, a storing section 7, a signal processing section 8, and a control section 9.

### [Configuration of the Operation Section]

The operation section 2 includes a plurality of buttons disposed on an exterior case of the measuring device 1 and outputs an operation signal corresponding to an input (pressed) button to the control section 9. For example, the operation section 2 outputs an operation signal corresponding to input operation for displaying a setting screen and an operation signal corresponding to setting operation for an item displayed on the setting screen. Note that the operation section 2 is not limited to a configuration including the buttons and may be a touch panel disposed on a display section 51 explained below or a configuration for detecting tap operation of the user.

### [Configuration of the Detecting Section]

The detecting section 3 includes a biological-information detecting section 31 that detects biological information of the user under control by the control section 9 and a body-motion-information detecting section 32 that detects body motion information of the user under the control by the control section 9.

The biological-information detecting section 31 detects the biological information of the user as a signal and outputs the detected signal to the control section 9. The biological-information detecting section 31 includes a pulse wave sensor that detects a pulse wave of the user. Besides, the biological-information detecting section 31 may include sensors that detect other biological information (e.g., a brain wave, an electrocardiogram, and a body temperature).

In this embodiment, the pulse wave sensor included in the biological-information detecting section 31 is a photoelectric sensor including a light emitting element configured by an LED (Light Emitting Diode) or the like and a first light receiving element and a second light receiving element respectively configured by photodiodes or the like. In the pulse wave sensor, the light emitting element radiates light toward an organism and the light receiving elements receive lights arriving through blood vessels of the organism. A pulse rate is counted by analyzing a pulse wave signal obtained by integrating signals indicating temporal changes of light amounts of the lights received by the light receiving elements. That is, the light radiated on the organism is partially absorbed by the blood vessels. Since an absorption ratio in the blood vessels changes because of the influence of pulsation, the light amounts of the light reaching the light receiving elements change. The signal processing section 8 explained below carries out a frequency analysis on the pulse wave signal obtained from the signals indicating the temporal changes of the received light amounts detected and output by the light receiving elements. A frequency of a pulse is specified from a result of the frequency analysis by the control section 9 explained below. A pulse rate (a pulse rate per unit time) of the user is calculated and measured on the basis of the frequency of the pulse.

The body-motion-information detecting section 32 detects and outputs body motion information of the user as a signal (a body motion signal). Specifically, the body-motion-information detecting section 32 includes an acceleration sensor that detects, as body motion information, an acceleration signal that changes according to a body motion of the user. The body-motion-information detecting section 32 outputs an acceleration signal indicating a change in a detected acceleration value to the control section 9 as the body motion signal. The acceleration sensor can be configured by a three-axis sensor that detects accelerations in orthogonal three axes in an X direction, a Y direction, and a Z direction.

When the measuring device 1 is worn on the wrist of the user, an acceleration signal of one axis among the orthogonal three axes is a signal indicating a swing of an arm of the user during exercise. An acceleration signal of another one axis is a signal indicating a pace (a pitch: the number of steps per one minute) of the user during exercise.

An acceleration signal of still another one axis is a signal obtained by combining the swing of the arm and the pace.

The acceleration signals are also used for processing for reducing, from a pulse wave signal detected by the pulse wave sensor, body motion noise superimposed on the pulse wave signal.

The body-motion-information detecting section 32 may include a gyro sensor that detects angular velocity instead of or in addition to the acceleration sensor.

Note that, in the following explanation, among body motion signals detected by the body-motion-information detecting section 32, a signal indicating, as one cycle, an acceleration change involved in one reciprocating motion of a wearing part on which the measuring device 1 is worn is represented as a first body motion signal. A signal indicating, as one cycle, each of acceleration changes respectively involved in movement in one direction and movement in the other direction in the one reciprocating motion is represented as a second body motion signal.

Specifically, the first body motion signal is an acceleration signal indicating, as one cycle, an acceleration change involved in one swing (one reciprocating motion) of the wrist, which is the wearing part of the measuring device 1. In other words, the first body motion signal is an acceleration signal, one cycle of which is an acceleration change corresponding to a motion of two steps.

The second body motion signal is an acceleration signal indicating, as one cycle, each of an acceleration change involved in movement in a forward direction and an acceleration change involved in movement in a backward direction in one swing of the wrist. Note that, during walking or during running, the left and right feet are moved in the forward direction once each in order according to forward and backward reciprocation of the wrist. That is, one foot is moved once in the forward direction according to one swing of the wrist in the forward direction or the backward direction. Therefore, the second body motion signal can be rephrased as an acceleration signal indicating the movements of the respective feet and is an acceleration signal, one cycle of which is an acceleration change corresponding to a motion of one step. Further, the second body motion signal can be considered an acceleration signal (a body motion signal) indicating a pace.

### [Configuration of the Receiving Section]

The receiving section 4 is equivalent to the position-information acquiring section that acquires position information indicating a present position of the measuring device 1 (position information indicating a present position of the user). The receiving section 4 can be adapted to a satellite positioning system such as a GPS (Global Positioning System) and configured to acquire position information indicating the present position on the basis of a radio wave received from a satellite. Note that, instead of the configuration, the receiving section 4 can be configured to calculate position information using a wireless radio wave for communication. The position information acquired by the receiving section 4 may be adopted as one parameter indicating a body motion of the user.

### [Configuration of the Notifying Section]

The notifying section 5 notifies the user of various kinds of information under the control by the control section 9. The notifying section 5 includes the display section 51, a sound output section 52, and a vibrating section 53.

Although not shown in the figure, the display section 51 is configured by display means such as various display panels of liquid crystal or the like and a plurality of LEDs. The display section 51 displays information input from the control section 9. For example, the display section 51 displays biological information and body motion information detected by the detecting section 3. The display section 51 displays, by causing the plurality of LEDs as indicators and lighting or flashing at least one of the plurality of LEDs, exercise intensity calculated on the basis of the biological information and the body motion information. Further, the display section 51 displays a setting screen for causing the user to input and set user information concerning the user. Note that, when a display size (a resolution) of an image on the display section 51 is small, the setting screen may be displayed in the information processing device.

The sound output section 52 includes sound output means such as a speaker and outputs sound corresponding to sound information input from the control section 9.

The vibrating section 53 includes a motor, the operation of which is controlled by the control section 9, and informs the user of for example, warning through vibration generated by driving of the motor.

### [Configuration of the Communication Section]

The communication section 6 includes a communication module capable of communicating with the information processing device. The communication section 6 transmits, for example, according to a request signal received from the information processing device, biological information and body motion information stored in the storing section 7 to the information processing device. When the user information is input to the setting screen displayed by the information processing device, the communication section 6 receives the user information from the information processing device.

Note that, in this embodiment, the communication section 6 communicates with an external device by radio according to a short-range wireless communication scheme. However, the communication section 6 may communicate with the external device via a relay device such as a cradle or may communicate with the external device via a cable.

### [Configuration of the Storing Section]

The storing section 7 is configured by storing means including a flash memory and stores various kinds of information. The storing section 7 includes a control-information storing section 71, a detected-information storing section 72, and a table storing section 73.

The control-information storing section 71 has stored therein control information such as various programs and data necessary for the operation of the measuring device 1.

The detected-information storing section 72 stores biological information (a pulse wave signal) and body motion information (a body motion signal) detected by the detecting section 3 and an analysis result (e.g., a pulse rate) by the signal processing section 8 and the control section 9 explained below. The detected-information storing section 72 sequentially stores these kinds of information. When a storage capacity runs short, the detected-information storing section 72 overwrites information stored earliest with information acquired anew.

The table storing section 73 has stored therein a plurality of tables for acquiring, when a pulse cannot be appropriately detected from the pulse wave signal, an estimated pulse rate corresponding to a degree of exercise carried out by the user. The tables are analysis information used in analyzing the pulse wave signal serving as the biological information and are respectively tables in which a pace indicating a degree of exercise and an estimated pulse rate are set in association with each other.

As users of the measuring device 1, besides a person not having exercise habits and a person having exercise habits (a person who carries out exercise every day), there is a person having a high cardiopulmonary function called athlete's heart. In these people, a pulse rate measured when exercise having the same pace is carried out is different. For example, when the exercise in the same pace is carried out, a pulse rate of the person not having exercise habits tends to be the highest and a pulse rate of the person having the athlete's heart tends to be the lowest. Therefore, it is difficult to acquire, from one table in which a cardiopulmonary function of a user is not taken into account, that is, one table in which a pace and a pulse rate are associated in a one-to-one relation, an estimated pulse rate corresponding to the user.

On the other hand, the table storing section 73 has stored therein three tables, that is, a table for ordinary people in which an estimated pulse rate of the person not having exercise habits is set with respect to a pace, a table for runners in which an estimated pulse rate of the person having exercise habits is set with respect to a pace, and a table for athlete's heart in which an estimated pulse rate of the person having the athlete's heart is set with respect to a pace. Note that the tables are selected by the control section 9 explained below on the basis of content of input operation by the user or detected biological information and body motion information. The selected tables are used in acquisition of an estimated pulse rate. Besides, the selected tables are corrected as appropriate according to a pace and a pulse rate calculated according to exercise of the user.

Note that, in this embodiment, the athlete's heart indicates a heart itself enlarged to be larger than usual as a result of the body of the user being adapted to an improved athletic capability or an excellent cardiopulmonary function obtained by having such a heart. Note that a condition for determining that a person is a person having an athlete's heart (hereinafter referred to as determination condition) is that, for example, a cardiac output per one stroke is 150 ml or more, more desirably, 200 ml or more or a resting pulse rate is 50 or less, more desirably, 40 or less. The cardiac output and the resting pulse rate can be extracted by analyzing a pulse wave or an electrocardiogram. The athlete's heart can be rephrased as exercise capability or cardiopulmonary capability. The determination condition can be set as, for example, time of a long-distance race.

### [Configuration of the Signal Processing Section]

The signal processing section 8 includes a signal processing circuit such as a DSP (Digital Signal Processor) and executes signal processing of biological information and body motion information detected by the detecting section 3.

Specifically, the signal processing section 8 executes processing for integrating respective signals input from the first light receiving element and the second light receiving element and generating a pulse wave signal. The signal processing section 8 determines an axis of the acceleration signal (the first body motion signal) indicating the swing of the arm of the user and an axis of the acceleration signal (the second body motion signal) indicating the pace of the user among the acceleration signals of the orthogonal three axes input from the acceleration sensor included in the body-motion-information detecting section 32. Since the two axes are determined, the remaining axis of acceleration indicating the swing of the arm and the pace of the user is also determined.

The signal processing section 8 removes body motion noise components (signals of noise components involved in the swing of the arm and the pace) from the generated pulse wave signal using an adaptive filter such as an FIR (Finite Impulse Response) filter. The signal processing section 8 performs a frequency analysis at a predetermined frequency such as FFT (Fast Fourier Transform) on a pulsation signal, which is the pulse wave signal from which the body motion noise components are removed. The signal processing section 8 outputs an obtained processing result to the control section 9. A frequency of a pulse is specified by the control section 9 according to a peak included in the processing result. A pulse rate per unit time is calculated.

As explained above, the pulse wave signal is detected by the pulse wave sensor included in the biological-information detecting section 31 and is a signal before the body motion noise components are removed. That is, in this embodiment, the pulse wave signal is a signal obtained by integrating signals respectively detected by the first light receiving element and the second light receiving element. Note that, when the pulse wave sensor includes one light receiving element, a signal detected by the light receiving element is the pulse wave signal. The body motion noise components are included in the pulse wave signal.

On the other hand, the pulsation signal is a signal obtained by removing the body motion noise components from the pulse wave signal with the adaptive filer. That is, the pulsation signal is the pulse wave signal from which the body motion noise components are removed.

### [Configuration of the Control Section]

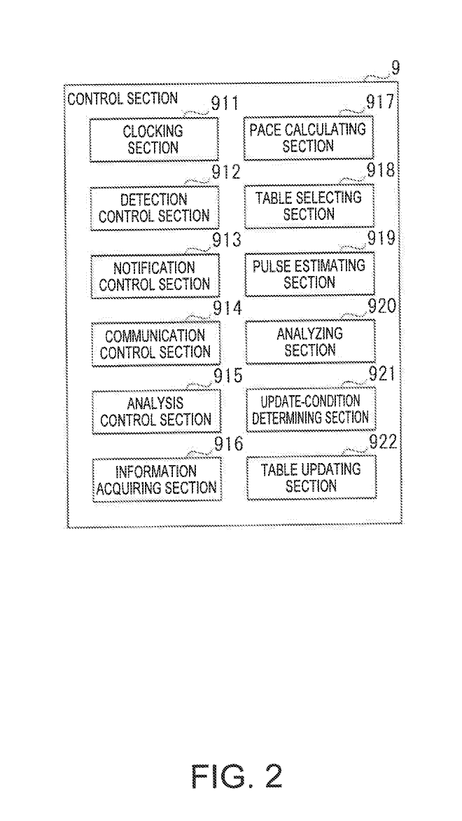

FIG. 2 is a block diagram showing the configuration of the control section 9.

The control section 9 includes an arithmetic processing circuit such as a CPU (Central Processing Unit) and controls the operation of the measuring device 1 autonomously or according to an operation signal input from the operation section 2. For example, the control section 9 causes the storing section 7 to store biological information and body motion information detected by the detecting section 3. The control section 9 causes the storing section 7 to store input information to the displayed setting screen. Besides, the control section 9 selects the table used for acquisition of an estimated pulse rate. Further, the control section 9 calculates a pulse rate on the basis of the processing result of the signal processing section 8. Besides, the control section 9 updates the selected table on the basis of a measured pulse rate.

In order to executes the processing explained above, the control section 9 includes, as functional sections realized by the arithmetic processing circuit executing the programs stored in the control-information storing section 71, as shown in FIG. 2, a clocking section 911, a detection control section 912, a notification control section 913, a communication control section 914, an analysis control section 915, an information acquiring section 916, a pace calculating section 917, a table selecting section 918, a pulse estimating section 919, an analyzing section 920, an update-condition determining section 921, and a table updating section 922.

### [Configurations of the Clocking Section, the Detection Control Section, and the Communication Control Section]

The clocking section 911 clocks present date and time.

The detection control section 912 controls the operation of the detecting section 3 and causes the detection section 3 to output a detection result by the detecting section 3 to the signal processing section 8. Besides, the detection control section 912 causes the detecting section 3 to store the detection result in the detected-information storing section 72 together with the present date and time.

The communication control section 914 controls the operation of the communication section 6 that communicates with the information processing device.

### [Configuration of the Notification Control Section]

The notification control section 913 controls the operation of the notifying section 5. For example, the notification control section 913 outputs notification information including display and sound indicating an operation state of the measuring device 1, a detection result by the detecting section 3, and the like to the notifying section 5 and causes the notifying section 5 to notify the notification information. The notification control section 913 drives the motor of the vibrating section 53 according to necessity and causes the notifying section 5 to notify predetermined information with vibration generated by the driving of the motor.

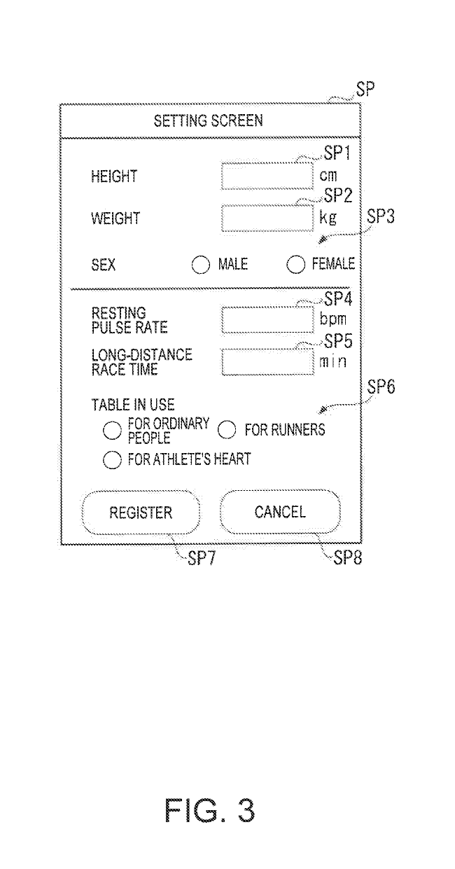

FIG. 3 is a diagram showing an example of a setting screen SP displayed by the display section 51.

Further, the notification control section 913 causes the display section 51 to display, for example, the setting screen SP shown in FIG. 3. The setting screen SP is a screen for causing the user to input the user information. On the setting screen SP, input fields SP1 and SP2 for causing the user to input the height and the weight of the user and a selection field SP3 for causing the user to select the sex of the user are set. Among the fields, the selection field SP3 is configured by two radio buttons inscribed with “male” and “female”. The selection field SP3 is configured such that the two radio buttons are not simultaneously selected.

On the setting screen SP, input fields SP4 and SP5 for inputting a resting pulse rate and time of a long-distance race (e.g., marathon) of the user and a selection field SP6 for causing the user to select a table corresponding to the user among the three tables used for estimation of a pulse rate are set. Among the fields, the selection field SP6 is configured by three radio buttons inscribed with “for ordinary people”, “for runners”, and “for athlete's heart”.

When a registration button SP7 provided in a lower part of the setting screen SP is pressed, input contents of the fields SP1 to SP6 are acquired by the information acquiring section 916 explained below and stored in the storing section 7. On the other hand, when a cancel button SP8 is pressed, a screen before shift to the setting screen SP is displayed.

Note that the setting screen SP may be configured such that information concerning age of the user can be set instead of or in addition to the user information. For example, an input field for causing the user to input age as such information or an input field for causing the user to input date of birth as such information may be provided. In the latter case, the control section 9 may calculate the age of the user on the basis of the input date of birth and the present date and time clocked by the clocking section 911.

In the setting screen SP, the fields SP4 to SP6 are not essential items. Therefore, when information is not input to the items, second selection processing is executed by the table selecting section 918 explained below. A table corresponding to a pulse rate during exercise among the three tables is selected. The second selection processing is explained in detail below.

Note that, as explained above, when a display size of an image by the display section 51 is small, the notification control section 913 may cause the information processing device to display the setting screen SP and receive user information from the information processing device via the communication section 6. The setting screen SP is not limited to the configuration shown in FIG. 3. The setting screen SP may be configured to switch a display screen for each of setting items and cause the user to input the user information on display screens. Further, items to be input are not limited to the items explained above.

### [Configuration of the Analysis Control Section]

Referring back to FIG. 2, the analysis control section 915 controls the operation of the signal processing section 8. For example, when the measuring device 1 is not worn on the user, the analysis control section 915 regulates the signal processing by the signal processing section 8 and suppresses power consumption. When input operation for starting detection of biological information and body motion information is performed (or when detecting that the measuring device 1 is worn on the user), the analysis control section 915 causes the signal processing section 8 to execute the signal processing. When calculating a pulse rate, the analysis control section 915 sometimes switches, with the pulsation signal and the pulse wave signal, an execution target of a frequency analysis by the signal processing section 8.

### [Configurations of the Information Acquiring Section and the Pace Calculating Section]

The information acquiring section 916 acquires various kinds of information input from the operation section 2, the detecting section 3, the receiving section 4, and the communication section 6. For example, the information acquiring section 916 causes the storing section 7 to store biological information and body motion information input from the detecting section 3 and position information input from the receiving section 4. The information acquiring section 916 acquires, on the basis of an operation signal input from the operation section 2 during the display of the setting screen SP, user information input on the setting screen SP.

The pace calculating section 917 calculates a pace of the user on the basis of a frequency analysis result with respect to the second body motion signal (an acceleration signal indicating a pace).

### [Configuration of the Table Selecting Section]

The table selecting section 918 selects a table corresponding to the user from the three tables stored in the table storing section 73. Specifically, the table selecting section 918 executes first selection processing for selecting, from the three tables, a table corresponding to the user information input on the setting screen SP. On the other hand, when input to the input fields SP4 and SP5 and selection in the selection field SP6 are not performed on the setting screen SP and a resting pulse rate and a long-distance race time of the user and a selected table by the user cannot be acquired, the table selecting section 918 executes second selection processing for selecting, from the three tables, a table corresponding to a pulse rate during exercise of the user.

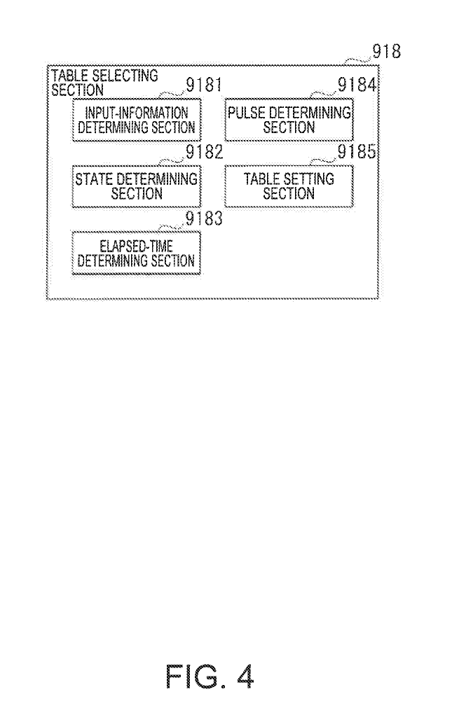

FIG. 4 is a block diagram showing the configuration of the table selecting section 918.

In order to execute the first selection processing and the second selection processing, the table selecting section 918 includes, as shown in FIG. 4, an input-information determining section 9181, a state determining section 9182, an elapsed-time determining section 9183, a pulse determining section 9184, and a table setting section 9185. Among the sections, the input-information determining section 9181 and the pulse determining section 9184 are equivalent to the user determining section that determines an exercise capability (a capability concerning a cardiopulmonary function) of the user in the table selecting section 918.

The input-information determining section 9181 determines user information input by the user on the setting screen SP and acquired by the information acquiring section 916.

Specifically, the input-information determining section 9181 determines on the basis of the user information whether the table for athlete's heart is selected in the selection field SP6 on the setting screen SP.

The input-information determining section 9181 determines whether time of a long-distance race input to the input field SP5 on the setting screen SP is within a predetermined time (time for enabling determination of the person having the athlete's heart; for example, three hours in the case of marathon).

Further, the input-information determining section 9181 determines whether a resting pulse rate input to the input field SP4 on the setting screen SP is smaller than an index value (e.g., a pulse rate of 40), which is an indicator of the person having the athlete's heart, and determines whether the resting pulse rate exceeds a predetermined value (e.g., a pulse rate of 70), which is an ordinary resting pulse rate of the person not having exercise habits.

The state determining section 9182 determines a state of the user on the basis of detected body motion information. Specifically, the state determining section 9182 determines whether the user is walking or running. The state determining section 9182 determines whether the state of the user is a resting state.

When the state determining section 9182 determines that the state of the user is the resting state, the elapsed-time determining section 9183 determines whether a predetermined time (e.g., one minute) has elapsed after the state of the user changes to the resting state.

The pulse determining section 9184 determines a pulse rate calculated on the basis of detected biological information and body motion information. Specifically, when the state determining section 9182 determines that the user is walking or running, the pulse determining section 9184 determines whether the pulse rate is smaller than a predetermined value. The predetermined value can be set to a value corresponding to a calculated pace of the user and can be set to, for example, a value obtained by adding an error pulse rate (e.g., 10 to 20) to a half value of the pace. For example, the predetermined value may be set to an estimated pulse rate corresponding to the pace among estimated pulse rates set in the table for athlete's heart or the table for runners.

When the elapsed-time determining section 9183 determines that the predetermined time has elapsed after the state of the user changes to the resting state, the pulse determining section 9184 determines whether a calculated pulse rate (i.e., a resting pulse rate) is smaller than the index value, which is the indicator of the person having the athlete's heart, and whether the pulse rate exceeds the predetermined value, which is the ordinary resting pulse rate of the person having exercise habits.

The table setting section 9185 is equivalent to the information setting section that sets a table serving as analysis information. The table setting section 9185 sets, on the basis of determination results by the determining sections 9181 to 9184, any one of the table for ordinary people, the table for runners, and the table for athlete's heart as a table used for analysis of biological information.

The first selection processing and the second selection processing executed by the table selecting section 918 to select a table serving as analysis information is explained below. Note that, as explained above, the first selection processing is the processing for selecting a table on the basis of user information input by the user. The second selection processing is the processing for selecting a table on the basis of detected biological information and body motion information.

### [First Selection Processing]

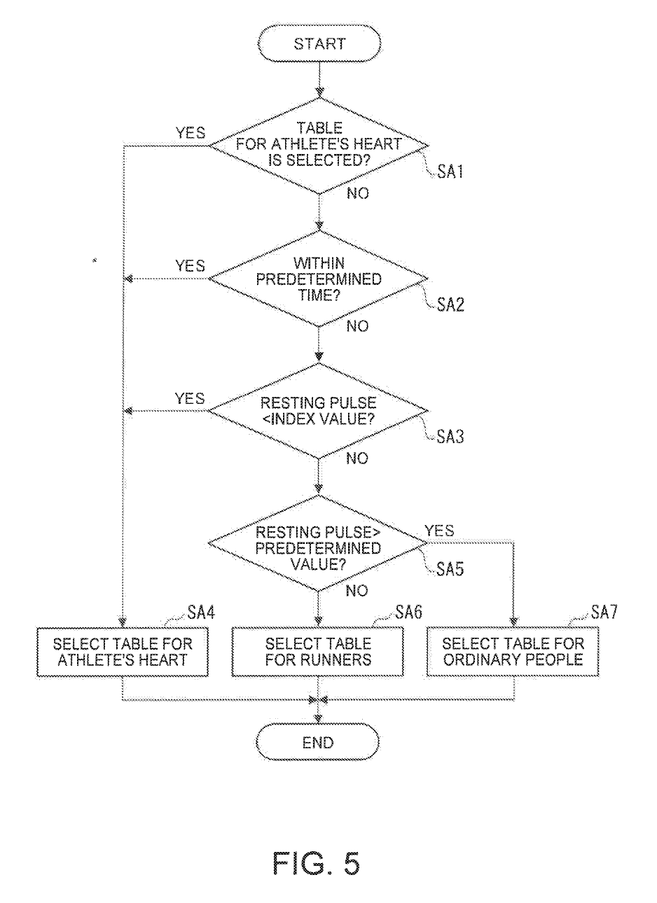

FIG. 5 is a flowchart showing the first selection processing.

When the registration button SP7 is pressed on the setting screen SP, the table selecting section 918 executes the first selection processing shown in FIG. 5.

In the first selection processing, first, the input-information determining section 9181 determines whether a table selected in the table selection field SP6 of the setting screen SP is the table for athlete's heart (whether input operation for selecting the table for athlete's heart as a table to be used is carried out by the user) (step SA1).

When determining that the table is the table for athlete's heart, the table selecting section 918 shifts the processing to step SA4.

On the other hand, when determining that the table is not the table for athlete's heart, the input-information determining section 9181 determines whether time input to the long-distance race time input field SP5 of the setting screen SP is within the predetermined time (step SA2).

When it is determined in the determination processing in step SA2 that the input long-distance race time is within the predetermined time, the table selecting section 918 shifts the processing to step SA4.

When it is determined in the determination processing in step SA2 that the input long-distance race time is not within the predetermined time, the input-information determining section 9181 determines whether a value input to the resting pulse rate input field SP4 of the setting screen SP is smaller than the index value (step SA3). That is, in step SA3, as in step SA2, the input-information determining section 9181 determines on the basis of information input by the user whether the user is the person having the athlete's heart.

When it is determined in the determination processing in step SA3 that the input resting pulse rate is smaller than the index value, the table selecting section 918 shifts the processing to step SA4.

In step SA4, the table setting section 9185 selects and sets, as a table used for estimation of a pulse rate, the table for athlete's heart among the three tables stored in the table storing section 73 (step SA4).

After step SA4, the table selecting section 918 ends the first selection processing.

On the other hand, when it is determined in the determination processing in step SA3 that the input resting pulse rate is not smaller than the index value, the pulse determining section 9184 determines whether the resting pulse rate exceeds the predetermined value (step SA5).

When it is determined in the determination processing in step SA5 that the input resting pulse rate does not exceeds the predetermined value (i.e., it is determined that the resting pulse rate is higher than a resting pulse rate of the person having the athlete's heart but is lower than a resting pulse rate of the person not having exercise habits), the table selecting section 918 determines that the user wearing the measuring device 1 is the person having exercise habits. In this case, the table setting section 9185 selects and sets, as the table used for estimation of a pulse rate, the table for runners among the three tables stored in the table storing section 73 (step SA6).

On the other hand, when it is determined in the determination processing in step SA5 that the input resting pulse rate exceeds the predetermined value, that is, the resting pulse rate is relatively high, the table selecting section 918 determines that the user wearing the measuring device 1 is the person not having exercise habits. In this case, the table setting section 9185 selects and sets, as the table used for estimation of a pulse rate, the table for ordinary people among the three tables stored in the table storing section 73 (step SA7).

After steps SA6 and SA7, the table selecting section 918 ends the first selection processing. According to the first selection processing, the table used for estimation of a pulse rate is selected on the basis of the user information input by the user.

### [Second Selection Processing]

When a table to be used is not selected and a resting pulse rate and a long-distance race time are not input on the setting screen SP, selection and setting of a table based on these kinds of user information cannot be executed. In this case, the table selecting section 918 executes the second selection processing for selecting, on the basis of detected biological information and body motion information, a table to be used. That is, the table selecting section 918 grasps an exercise capability of the user on the basis of an exercise state of the user based on the detected body motion information and biological information of the user in the exercise state and executes the second selection processing for selecting and setting a table, which is analysis information, corresponding to the exercise capability. The second selection processing may be executed while a message or the like for urging exercise or a rest is notified and the user carries out an action corresponding to the message. Alternatively, the second selection processing may be executed targeting biological information and body motion information already stored in the storing section 7.

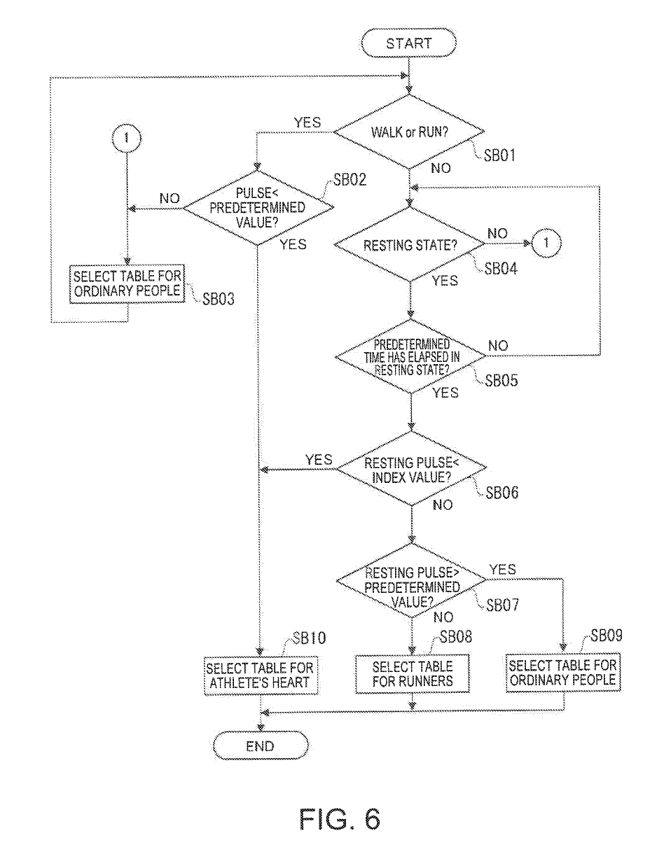

FIG. 6 is a flowchart showing the second selection processing.

In the second selection processing, as shown in FIG. 6, first, the state determining section 9182 determines on the basis of a pace calculated by the pace calculating section 917 whether the user is walking or running (whether the user is exercising) (step SB01). Note that, when the second selection processing is executed targeting biological information (a pulse wave signal) and body motion information (a body motion signal) already stored, in step SB01, the state determining section 9182 determines on the basis of the calculated pace whether the body motion information and the biological information are body motion information and biological information during exercise.

When it is determined in the determination processing in step SB01 that the user is walking or running (when it is determined that processing target is the biological information and the body motion information during walking or during running), the pulse determining section 9184 determines whether a pulse rate calculated on the basis of the biological information and the body motion information is smaller than the predetermined value (a value corresponding to the pace) (step SB02). That is, in step SB02, the pulse determining section 9184 determines an exercise capability of the user on the basis of an exercise state based on the body motion information of the user and a pulse rate based on the biological information and the body motion information and determines whether the user is the person having the athlete's heart.

When it is determined in step SB02 that the calculated pulse rate is not smaller than the predetermined value, the table setting section 9185 temporarily selects and sets the table for ordinary people as the table used for estimation of a pulse rate (step SB03). The table selecting section 918 returns the processing to step SB01 and executes the second selection processing again. Note that the table selected and set in step SB03 may be the table for runners.

On the other hand, when it is determined in the determination processing in step SB02 that the calculated pulse rate is smaller than the predetermined value, the table selecting section 918 shifts the processing to step SB10.

When it is determined in the determination processing in step SB01 that the user is not walking or running (when it is determined that the processing target is not the biological information and the body motion information during the waking or the running), the state determining section 9182 determines on the basis of the biological information and the body motion information whether a state of the user is a resting state (step SB04).

When it is determined in the determination processing in step SB04 that the state of the user is not the resting state, the table selecting section 918 shifts to step SB03. The table setting section 9185 temporarily selects and sets the table for ordinary people as the table to be used and returns the processing to step SB01.

When it is determined in the determination processing in step SB04 that the state of the user is the resting state, the table selecting section 918 determines whether the predetermined time has elapsed after the state of the user changes to the resting state (step SB05).

When it is determined in the determination processing in step SB05 that the predetermined time has not elapsed, the table selecting section 918 returns the processing to step SB04.

On the other hand, when it is determined in the determination processing in step SB05 that the predetermined time has elapsed, the pulse determining section 9184 sets, as a resting pulse rate, a pulse rate calculated on the basis of the biological information and the body motion information and determines whether the resting pulse rate is smaller than the index value (the index value used in the determination processing in step SA3) (step SB06).

When it is determined in the determination processing in step SB06 that the resting pulse rate is smaller than the index value, the table selecting section 918 shifts the processing to step SB10.

On the other hand, when it is determined in the determination processing in step SB06 that the resting pulse rate is not smaller than the index value, the pulse determining section 9184 determines whether the resting pulse rate exceeds the predetermined value (the predetermined value used in the determination processing in step SA5) (step SB07).

When it is determined in the determination processing in step SB07 that the resting pulse rate does not exceed the predetermined value, as in step SA6, the table setting section 9185 selects and sets the table for runners as the table to be used (step SB08).

On the other hand, when it is determined in the determination processing in step SB07 that the resting pulse rate exceeds the predetermined value, the table setting section 9185 selects and sets the table for ordinary people as the table to be used (step SB09).

In step SB10, the table setting section 9185 selects and sets the table for athlete's heart as the table to be used (step SB10).

After steps SB08 to SB10, the table selecting section 918 ends the second selection processing. According to the second selection processing, the table used for estimation of a pulse rate is selected on the basis of the detected biological information and body motion information.

In the first selection processing and the second selection processing, the table selecting section 918 automatically selects and sets the table corresponding to the user on the basis of the input user information or the detected biological information (pulse wave signal) and body motion information (body motion signal). However, the table selecting section 918 is not limited to this and may urge the user to change the table by, for example, displaying a message for causing the user to select the table. Only one of the first selection processing and the second selection processing may be executed. Further, the second selection processing may be periodically executed.

### [Configuration of the Pulse Estimating Section]

Referring back to FIG. 2, the pulse estimating section 919 estimates a pulse rate of the user on the basis of the detected body motion information (body motion signal). Specifically, the pulse estimating section 919 refers to the table selected by the table selecting section 918 and acquires, as an estimated pulse rate, a pulse rate corresponding to the pace calculated on the basis of the second body motion signal by the pace calculating section 917.

### [Configuration of the Analyzing Section]

The analyzing section 920 analyzes detected biological information. Specifically, the analyzing section 920 calculates a pulse rate on the basis of a result of the frequency analysis by the signal processing section 8.

The measuring device 1 sets, as a frequency of a pulse, a representative frequency (a frequency having a large peak) in a result (a power spectrum) of the frequency analysis for the pulsation signal and sets, as a pulse rate, a value obtained by multiplying the calculated frequency with 60. In such a configuration, when the frequency of the body motion (the swing of the arm and the pace) and the frequency of the pulse are close, body noise components are excessively subtracted from the pulse wave signal when the pulsation signal is calculated in the adaptive filter processing by the signal processing section 8. Therefore, the peak of the frequency of the pulse in the power spectrum serving as the result of the frequency analysis of the pulsation signal sometimes decreases (weakens). Besides, even when respective phases of the body motion and the pulse are reversed, the peak decrease (weakens). In such a case, in some case, a peak indicating occurrence timing of a pulse in the result of the frequency analysis (the peak of the frequency of the pulse) sometimes decreases and the pulse cannot be specified. Besides, when a peak deriving from noise is detected in the frequency of the pulse, the peak is likely to be erroneously determined as a pulse.

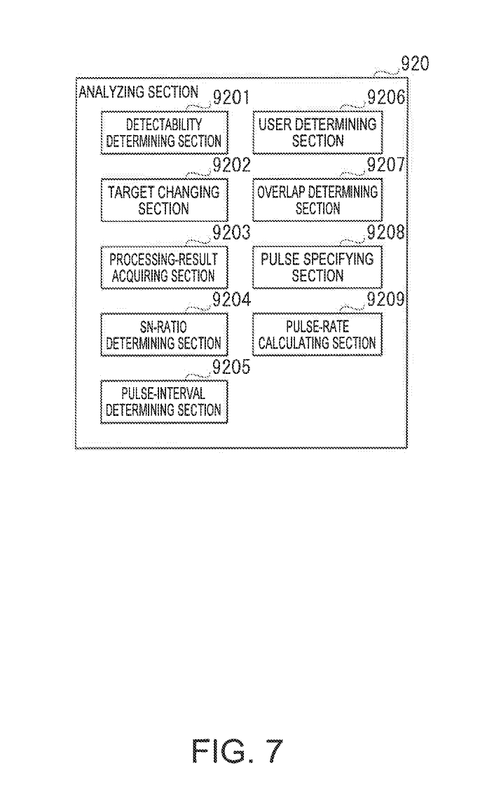

FIG. 7 is a block diagram showing the configuration of the analyzing section 920.

Therefore, the analyzing section 920 detects or specifies a pulse on the basis of the pulsation signal (more specifically, the power spectrum obtained by performing a frequency analysis of the pulsation signal) at normal time. However, when a pulse cannot be detected and specified on the basis of the pulsation signal, as explained in detail below, the analyzing section 920 determines whether the pulse wave signal and the body motion signal overlap. When determining that the pulse wave signal and the body motion signal overlap, the analyzing section 920 grasps the body motion as a pulse.

In order to execute such processing, the analyzing section 920 includes, as shown in FIG. 7, a detectability determining section 9201, a target changing section 9202, a processing-result acquiring section 9203, an SN-ratio determining section 9204, a pulse-interval determining section 9205, a user determining section 9206, an overlap determining section 9207, a pulse specifying section 9208, and a pulse-rate calculating section 9209.

The detectability determining section 9201 determines whether a pulse can be detected (specified) from the pulsation signal. Specifically, the detectability determining section 9201 determines whether a peak equivalent to a pulse can be detected from a peak change in a predetermined frequency band of the power spectrum calculated from the pulsation signal.

When the detectability determining section 9201 determines that a pulse cannot be detected from the pulsation signal, the target changing section 9202 causes the signal processing section 8 to execute the frequency analysis on the pulse wave signal instead of the pulsation signal.

The processing-result acquiring section 9203 acquires the result (the power spectrum) of the frequency analysis of the pulse wave signal by the signal processing section 8.

The SN-ratio determining section 9204 determines whether an SN ratio of the pulse wave signal is satisfactory, specifically, whether the SN ratio is equal to or larger than a predetermined value. The predetermined value can be set to a value of a degree at which body motion noise components included in the pulse wave signal are determined to be sufficiently low.

The pulse-interval determining section 9205 determines whether a difference between the last pulse, which is a detected or estimated pulse rate of the last time, and a pulse candidate (i.e., a candidate of a present pulse rate) selected as a pulse rate after the last pulse is within a specified difference. When it is determined in the determination processing that the difference between the last pulse and the pulse candidate is within the specified difference, the pulse candidate is highly likely to be an actual present pulse. In other words, when it is determined that the difference between the last pulse and the pulse candidate is not within the specified difference, it is highly likely that the pulse candidate is not the actual present pulse and the measuring device 1 loses sight of the pulse (the pulse wave signal).

The user determining section 9206 determines on the basis of a processing result by the table selecting section 918 whether the user is the person having the athlete's heart. For example, when the table selected by the table selecting section 918 is the table for athlete's heart, the user determining section 9206 determines that the user is the person having the athlete's heart. Note that the user determining section 9206 may determine on the basis of the user information independently from the selection result by the table selecting section 918 whether the user is the person having the athlete's heart. The user determining section 9206 may execute processing same as the determination processing by the table selecting section 918 and determine on the basis of a pulse rate during exercise or during a rest of the user whether the user is the person having the athlete's heart.

The overlap determining section 9207 determines on the basis of a processing result acquired by the processing-result acquiring section 9203 whether the pulse wave signal and the body motion signal overlap. Note that the pulse wave signal and the body motion signal overlap when main frequencies of the body motion signal and the pulse wave signal are the same or close to each other as a result of respective frequency analyses of the body motion signal and the pulse wave signal. That is, the pulse wave signal and the body motion signal overlap when a difference between the frequency of the body motion (body motion related information) and the frequency of the pulse (pulse related information) is within a predetermined range (e.g., a range of −0.1 Hz or more and +0.1 Hz or more).

Specifically, when the pulse-interval determining section 9205 determines that the difference between the last pulse and the pulse candidate is not within the specified difference, the overlap determining section 9207 compares the frequency of the last pulse specified from the frequency analysis result of the pulse wave signal and the frequency of the body motion specified from the second body motion signal. The overlap determining section 9207 carries out determination processing for determining whether a difference between the frequency of the last pulse and the frequency of the body motion is within the predetermined range.

When the difference between the frequency of the last pulse and the frequency corresponding to the second body motion signal (the frequency of the present body motion) is within the predetermined range, it is highly likely that the pulse wave signal and the body motion signal overlap. Therefore, when the difference between the frequencies is within the predetermined range, the overlap determining section 9207 estimates that the pulse wave signal and the second body motion signal overlap and a pulse of the present pulse is lost sight of. On the other hand, when the difference between the frequencies is not within the predetermined range, the overlap determining section 9207 estimates that the pulse wave signal and the second body motion signal do not overlap and the pulse of the present pulse is lost sight of because of other reasons.

When the user determining section 9206 determines that the user is the person having the athlete's heart, the overlap determining section 9207 compares the frequency of the last pulse specified from the frequency analysis result of the pulse wave signal and the frequency of the body motion specified from the first body motion signal (a frequency corresponding to the first body motion signal). The overlap determining section 9207 carries out determination processing for determining whether the difference between the frequency of the last pulse and the frequency of the body motion is within the predetermined range. In this case as well, when the difference between the frequencies is within the predetermined range, the overlap determining section 9207 estimates that the pulse wave signal and the first body motion signal overlap. When the difference between the frequencies is not within the predetermined range, the overlap determining section 9207 estimates that the pulse wave signal and the first body motion signal do not overlap.

Note that, in the respective kinds of determination processing, the overlap determining section 9207 may adopt a frequency of a pulse calculated from the estimated pulse rate instead of the frequency of the last pulse. The frequency can be calculated by dividing, by 60, an estimated pulse rate acquired from a currently selected table according to a present pace of the user calculated by the pace calculating section 917. In this case, in the following processing, the frequency of the last pulse only has to be replaced with the frequency of the estimated pulse rate.

The pulse specifying section 9208 specifies a peak of the pulse.

Specifically, when the detectability determining section 9201 determines that a pulse can be detected from the pulsation signal, the pulse specifying section 9208 specifies a pulse from a frequency analysis result of the pulsation signal.

When the overlap determining section 9207 determines that the difference between the frequency of the last pulse and the frequency of the body motion indicated by the second body motion signal is within the predetermined range and the pulse wave signal and the second body motion signal overlap, the pulse specifying section 9208 specifies the body motion as a pulse.

Further, when the overlap determining section 9207 determines that the difference between the frequency of the last pulse and the frequency of the body motion indicated by the first body motion signal is within the predetermined range and the pulse wave signal and the first body motion signal overlap, the pulse specifying section 9208 specifies the body motion as a pulse.

As explained above, the pulse-rate calculating section 9209 calculates, as a pulse rate, a value obtained by multiplying the frequency of the pulse specified by the pulse specifying section 9208 with 60. Note that, when the SN-ratio determining section 9204 determines that the SN ratio of the pulse wave signal is lower than the predetermined value, when the overlap determining section 9207 determines that the pulse wave signal and the second body motion signal do not overlap and the user determining section 9206 determines that the user is not the person having the athlete's heart, or when the overlap determining section 9207 determines that the pulse wave signal and the first body motion signal do not overlap, the pulse-rate calculating section 9209 acquires, as a present pulse rate, the estimated pulse rate acquired by the pulse estimating section 919.

The analyzing section 920 counts a pulse of the user with the functional sections 9201 to 9209 explained above. However, a detailed procedure of the pulse counting is explained in detail below.

### [Configuration of the Update-Condition Determining Section]

The update-condition determining section 921 determines on the basis of a pulse rate calculated by the analyzing section 920 whether update conditions for determining whether to update a table selected by the table selecting section 918 are satisfied. Examples of the update conditions include five conditions explained below. When all of the five conditions are satisfied, the update-condition determining section 921 determines that the update conditions for the table are satisfied.

A first condition is that a pace calculated by the pace calculating section 917 is stable for a predetermined time (e.g., 80 seconds) or more. Specifically, the first condition is that a change in a pace calculated in a predetermined period until the determination processing by the update-condition determining section 921 is executed is within a range of a predetermined value.

A second condition is that a pulse is continuously detected and specified a predetermined number of times (e.g., twenty times) or more on the basis of the pulsation signal. This is for the purpose of preventing a table from being updated according to an estimated pulse rate when a pulse is not appropriately detected, for example, when a wearing state of the measuring device 1 is bad.

A third condition is that an SN ratio of the detected pulse wave signal and an SN ratio of the pulsation signal are relatively high.

A fourth condition is that fluctuation in a calculated pulse rate is relatively small.

A fifth condition is that a frequency of a body motion and a frequency of a pulse are relatively apart from each other or a pulse rate calculated on the basis of the pulse wave signal is close to a value of a selected table. If one of the former condition and the latter condition is satisfied, the fifth condition is satisfied.

Note that, when the user is determined as the person having the athlete's heart, the update-condition determining section 921 determines whether a sixth condition explained below is satisfied in addition to the update conditions.

The sixth condition is that a pulse rate calculated by the pulse-rate calculating section 9209 is not a half or less of a pace of the user calculated by the pace calculating section 917 (in other words, the calculated pulse rate exceeds the half of the calculated pace of the user). This is because, when a user having an athlete's heart performs low-intensity exercise with which a pulse rate increases to only approximately a half of a pace, it is conceivable that a table in which a relation of a pace and a pulse rate matches a state of the user is not selected or a correct pulse rate cannot be acquired and the table is not appropriately updated.

### [Configuration of the Table Updating Section]

When the update-condition determining section 921 determines that the update conditions are satisfied, the table updating section 922 updates, in a selected table, a pulse rate of a corresponding pace with a pulse rate calculated by the pulse-rate calculating section 9209. When the update conditions are satisfied, the table updating section 922 updates the table according to an actual pulse rate of the user. Therefore, content of the table can be set to content corresponding to a relation between an actual pace and an actual pulse rate of the user. Note that the update of the table is not limited to the update for overwriting the pulse rate with the calculated pulse rate and can be update for overwriting the pulse rate with an average of a pulse rate in the past and a pulse rate calculated anew.

### [Pulse Rate Measurement Processing]

Since the measuring device 1 is worn on the wrist of the user, an acceleration change corresponding to a swing of the arm of the user appears in the first body motion signal and an acceleration change corresponding to a pace of the user appears in the second body motion signal. That is, the first body motion signal indicates a body motion component equivalent to one reciprocation of an arm swing during walking and during running of the user. The second body motion signal indicates a body motion component equivalent to one arm swing (i.e., one step) during walking and during running of the user.

When the body motion signals and the pulse wave signal overlap, as explained above, a pulse sometimes cannot be specified. When a peak deriving from noise is detected in a frequency of the pulse, there is a problem in that the peak is erroneously detected as a pulse.

Therefore, the control section 9 executes pulse rate measurement processing explained below in order to measure an appropriate pulse rate.

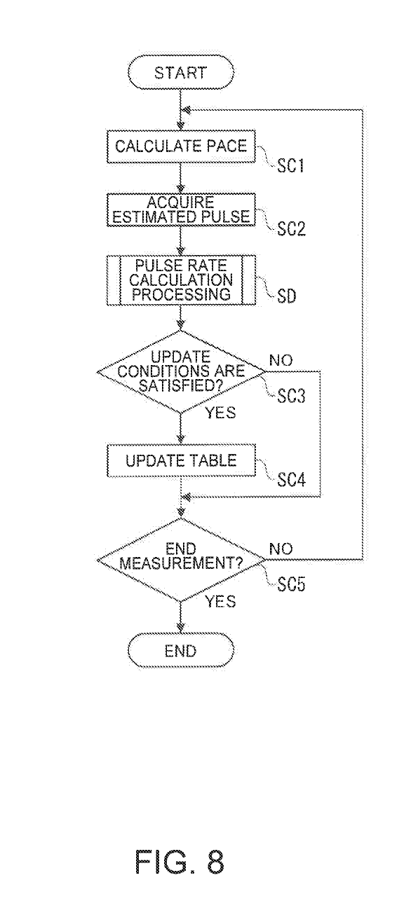

FIG. 8 is a flowchart showing the pulse rate measurement processing.

In the pulse rate measurement processing, as shown in FIG. 8, first, the pace calculating section 917 calculates a pace on the basis of detected body motion information (in particular, the second body motion signal) (step SC1).

Subsequently, the pulse estimating section 919 refers to a table selected by the table selecting section 918 and acquires a pulse rate (an estimated pulse rate) corresponding to the calculated pace (step SC2).

After step SC2, the control section 9 executes pulse rate calculation processing SD.

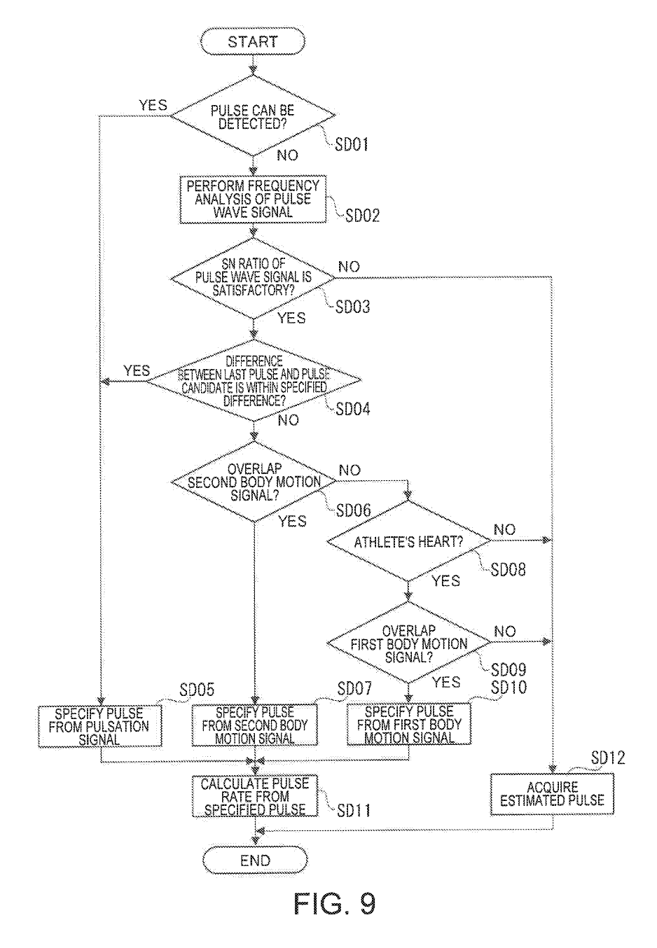

FIG. 9 is a flowchart showing the pulse rate calculation processing SD.

In the pulse rate calculation processing SD, as shown in FIG. 9, first, the detectability determining section 9201 determines whether a pulse can be detected and specified from the pulsation signal (step SD01).

When it is determined in the determination processing in step SD01 that a pulse can be detected, the control section 9 shifts the processing to step SD05.

On the other hand, when it is determined in the determination processing in step SD01 that a pulse cannot be detected from the pulsation signal, the target changing section 9202 causes the signal processing section 8 to execute a frequency analysis for the pulse wave signal. The processing-result acquiring section 9203 acquires a processing result of the frequency analysis (step SD02).

After step SD02, the SN-ratio determining section 9204 determines whether an SN ratio of the pulse wave signal is higher than a predetermined value (step SD03).

When it is determined that the SN ratio of the pulse wave signal is lower than the predetermined value, the control section 9 shifts the processing to step SD12.

On the other hand, when it is determined that the SN ratio of the pulse wave signal is higher than the predetermined value, the pulse-interval determining section 9205 determines whether an interval between the last pulse and the pulse candidate is within the specified interval (step SD04).

When it is determined that the interval is within the specified interval, the control section 9 shifts the processing to step SD05.

Note that the order of processing of steps SD02 and SD03 may be reversed. In this case, when it is determined that the SN ratio of the pulse wave signal is higher than the predetermined value, a frequency analysis for the pulse wave signal is executed. Thereafter, the processing only has to be shifted to step SD04.

In step SD05, the pulse specifying section 9208 specifies a pulse from the pulsation signal (step SD05). Thereafter, the control section 9 shifts the processing to step SD11.

When it is determined in the determination processing in step SD04 that the interval is not within the specified interval, the overlap determining section 9207 determines whether the pulse wave signal and the second body motion signal overlap, that is, whether a difference between a frequency of the last pulse and a frequency of a body motion indicated by the second body motion signal is within the predetermined range (step SD06).

When it is determined in the determination processing in step SD06 that the pulse wave signal and the second body motion signal overlap (the difference between the frequencies is within the predetermined range), the pulse specifying section 9208 specifies the body motion of the second body motion signal as a pulse (step SD07). Thereafter, the control section 9 shifts the processing to step SD11.

On the other hand, when it is determined in the determination processing in step SD06 that the pulse wave signal and the second body motion signal do not overlap (the difference between the frequencies is not within the predetermined range), the user determining section 9206 determines whether the user is the person having the athlete's heart (step SD08).

When it is determined in the determination processing in step SD08 that the user is not the person having the athlete's heart, the control section 9 shifts the processing to step SD12.

When it is determined in the determination processing in step SD08 that the user is the person having the athlete's heart, the overlap determining section 9207 determines whether the pulse wave signal and the first body motion signal overlap, that is, a difference between a frequency of the last pulse and a frequency of a body motion indicated by the first body motion signal is within the predetermined range (step SD09).

When it is determined in the determination processing in step SD09 that the pulse wave signal and the first body motion signal do not overlap (the difference between the frequencies is not within the predetermined range), the control section 9 shifts the processing to step SD12.

When it is determined in the determination processing in step SD09 that the pulse wave signal and the first body motion signal overlap (the difference between the frequencies is within the predetermined range), the pulse specifying section 9208 specifies the body motion of the first body motion signal as a pulse (step SD10). Thereafter, the control section 9 shifts the processing to step SD11.

Note that the frequency of the pulse compared with the frequency of the body motion in steps SD06 and SD09 may be a frequency of a pulse calculated from an estimated pulse rate as explained above.

In step SD11, the pulse-rate calculating section 9209 calculates a pulse rate on the basis of a frequency of the pulse specified in steps SD05, SD07, and SD10 (including a frequency of the body motion specified as the pulse) (step SD11).

On the other hand, in step SD12, the pulse-rate calculating section 9209 acquires an estimated pulse rate acquired from a table by the pulse estimating section 919 (step SD12).

After steps SD11 and SD12, the control section 9 ends the pulse rate calculation processing SD.

Referring back to FIG. 8, when the pulse rate calculation processing SD is ended, the update-condition determining section 921 determines whether the update conditions are satisfied (step SC3).

When it is determined that the update conditions are not satisfied, the control section 9 shifts the processing to step SC5.

On the other hand, when it is determined that the update conditions are satisfied, the table updating section 922 updates a currently selected table on the basis of the calculated pulse rate and the calculated pace (step SC4). Thereafter, the control section 9 shifts the processing to step SC5.

In step SC5, the control section 9 determines whether input operation for ending the measurement processing is performed by the user, that is, whether an operation signal corresponding to the input operation is input from the operation section 2 (step SC5).

When it is determined in the determination processing in step SC5 that the input operation is not performed, the control section 9 returns the processing to step SC1 and continues the pulse rate measurement processing.

On the other hand, when it is determined in the determination processing in step SC5 that the input operation is performed, the control section 9 ends the pulse rate measurement processing.

Since the pulse rate measurement processing is executed, it is possible to appropriately detect a pulse of the user and appropriately count and measure a pulse rate of the user.

### Effect of the First Embodiment

The measuring device 1 according to this embodiment explained above has an effect explained below.

When it is determined by the determination processing of the determining sections 9181 to 9184 that the exercise capability of the user satisfies the conditions and the user is the person having the athlete's heart, the table setting section 9185 selects and sets the table for athlete's heart as the table serving as the analysis information. The pulse estimating section 919 acquires an estimated pulse rate corresponding to a pace using the table. The analyzing section 920 retains and stores the estimated pulse rate as a pulse rate of the user. Consequently, when the user is the person having the athlete's heart, even if a pulse cannot be specified, it is possible to acquire a pulse rate on the basis of the table for athlete's heart. Therefore, it is possible to execute analysis processing of biological information corresponding to the exercise capability of the user and retain and notify an appropriate pulse rate. Therefore, it is possible to analyze and measure biological information appropriately for the user.

As explained above, the person having the athlete's heart is a person having an excellent cardiopulmonary function. The determining sections 9181 to 9184 determine whether the user is the person having the athlete's heart. A table corresponding to determination results of the determining sections 9181 to 9184 is selected and set. Therefore, biological information is analyzed on the basis of a table corresponding to a capability concerning the cardiopulmonary function of the user and a pulse rate is calculated. Consequently, it is possible to more appropriately calculate a pulse rate related to the cardiopulmonary function of the user. Therefore, it is possible to more appropriately analyze and measure the biological information.

As explained above, the person having the athlete's heart has a low resting pulse rate compared with the ordinary person (e.g., the person not having exercise habits). The pulse determining section 9184 determines on the basis of a resting pulse rate of the user whether the user is the person having the athlete's heart. Consequently, it is possible to appropriately determine whether the user is the person having the athlete's heart.

The input-information determining section 9181 determines on the basis of content of input operation on the setting screen SP whether the user is the person having the athlete's heart. Consequently, compared with when determination processing for determining whether the user is the person having the athlete's heart is executed by, for example, analyzing biological information and body motion information, it is possible to simply execute the determination processing.

The input-information determining section 9181 determines on the basis of not only a type of a table selected in the selection field SP6 of the setting screen SP displayed on the display section 51 but also time of a long-distance race and a resting pulse rate input to the input fields SP4 and SP5 whether the user is the person having the athlete's heart. Consequently, it is possible to execute the determination processing on the basis of these kinds of user information. Therefore, it is possible to more simply and appropriately perform the determination processing.

The pulse determining section 9184 compares a predetermined value corresponding to a pace during exercise of the user and a calculated pulse rate to determine whether the user is the person having the athlete's heart. Consequently, it is possible to appropriately determine on the basis of an actually detected pulse rate during exercise whether the user is the person having the athlete's heart.

The analyzing section 920 functioning as the analyzing section that analyzes detected biological information calculates a pulse rate of the user on the basis of the biological information. Consequently, even if the user is the person having the athlete's heart, it is possible to appropriately measure a pulse rate of the user. Therefore, it is possible to surely grasp fluctuation in the pulse rate that is characteristic of the person having the athlete's heart.

The analyzing section 920 acquires a pulse rate on the basis of a table in which a pace serving as an exercise state based on body motion information and a pulse rate are associated. Consequently, for example, when a body motion of the user is intense during exercise and biological information cannot be appropriately detected, it is possible to estimate a pulse rate of the user. Therefore, it is possible to present a pulse rate corresponding to the exercise state to the user.

Apulse rate corresponding to a pace of the user calculated by the pace calculating section 917 is acquired on the basis of a selected table. Consequently, even when a pulse cannot be specified from the pulsation signal and the pulse wave signal, it is possible to simply estimate a pulse rate corresponding to a pace of exercise being carried out. Therefore, it is possible to more simply present the pulse rate corresponding to the exercise state to the user.

When the overlap determining section 9207 determines that the pulse wave signal and the body motion signal overlap, the pulse specifying section 9208 specifies a body motion indicated by the body motion signal as a pulse. The pulse-rate calculating section 9209 calculates a pulse rate on the basis of a frequency of the body motion (body motion related information related to periodicity of the body motion). Consequently, when the pulse wave signal and the body motion signal overlap and a pulse cannot be specified from the pulsation signal obtained by removing the body motion signal from the pulse wave signal, it is possible to specify the body motion as a pulse and calculate a pulse rate on the basis of the frequency of the body motion. Therefore, it is possible to appropriately calculate the pulse rate and suppress noise from being erroneously detected as a pulse.

The measuring device 1 multiplies the frequency of the specified pulse with a coefficient corresponding to a unit time (60 if the unit time is one minute) to calculate a pulse rate per unit time. Consequently, compared with a method of counting a pulse rate within a predetermined time, multiplying the pulse rate with a quotient obtained by dividing the unit time by the predetermined time, and calculating a pulse rate per unit time, it is possible to quickly calculate the pulse rate.

In the case of the configuration explained above, a frequency of a pulse is acquired from a result of a frequency analysis for the pulsation signal obtained by removing body motion noise components from the pulse wave signal. Therefore, it is unnecessary to separately provide a configuration for calculating the frequency of the pulse. Besides, it is possible to acquire a frequency of a body motion using the configuration. Therefore, it is possible to acquire the respective frequencies of the pulse and the body motion using the configuration and the information used for the calculation of the pulse rate per unit time.

When it is determined that a difference between the frequency of the pulse and the frequency of the body motion is within a predetermined range, the overlap determining section 9207 determines that the pulse and the body motion overlap. Consequently, on the basis of a frequency of a pulse and a frequency of a body motion acquired from an analysis result by the signal processing section 8, it is possible to easily determine overlap of the pulse and the body motion.

When the difference between the frequency of the pulse and the frequency of the body motion is in a range of −0.1 Hz or more and +0.1 Hz or less, the overlap determining section 9207 determines that the pulse and the body motion overlap.

When the difference between the frequencies is outside the range, it is conceivable that the pulse wave signal and the body motion signal do not overlap and the influence of the body motion is small. Therefore, it is possible to appropriately determine the overlap of the pulse wave signal and the body motion signal by determining whether the difference is in the range of −0.1 Hz or more and +0.1 Hz or less.

When a frequency of the last pulse and a frequency corresponding to the body motion signal (a frequency of a present body motion) are within the range, it is highly likely that a pulse cannot be specified from the pulse wave signal because the pulse wave signal and the body motion signal overlap.

When a frequency of a pulse calculated from an estimated pulse rate and the frequency corresponding to the body motion signal are within the range, it is also highly likely that a pulse cannot be specified from the pulse wave signal because the pulse wave signal and the body motion signal overlap.

Therefore, by determining whether a difference between the frequency of the pulse calculated from the pulse rate of the last pulse or the estimated pulse rate and the frequency corresponding to the body motion signal is within the range, the overlap determining section 9207 can specify why a pulse cannot be specified from the pulse wave signal. When a pulse cannot be specified from the pulse wave signal because of the overlap of the pulse wave signal and the body motion signal, since a pulse rate is calculated on the basis of the frequency of the body motion, it is possible to more appropriately count the pulse rate.

When an SN ratio of the pulse wave signal is relatively high, the influence of noise such as a body motion component is small in the pulse wave signal. Even when the pulse wave signal and the body motion signal overlap, it is highly likely that a pulse can be specified. On the other hand, when the SN ratio of the pulse wave signal is relatively low, it is highly likely that a pulse cannot be specified. In such a case, it is difficult to calculate a pulse rate.

On the other hand, when the SN-ratio determining section 9204 determines that the SN ratio of the pulse wave signal is lower than a predetermined value, an estimated pulse rate corresponding to a pace calculated from the second body motion signal is acquired from a selected table. Consequently, even when a pulse cannot be detected and specified, it is possible to acquire and present a pulse rate estimated according to an exercise state of the user.

In general, a type of the body motion signal easily overlapping the pulse wave signal is sometimes different depending on exercise habits, an exercise capability, and the like of an individual. Specifically, in the person not having exercise habits, a result is obtained that a frequency of a body motion based on the second body motion signal and a frequency of a pulse based on the pulse wave signal are located in substantially the same frequency domain. In this case, it is determined that the pulse wave signal and the second body motion signal overlap.

On the other hand, in the person having exercise habits and having the athlete's heart, a result is obtained that a frequency of a body motion based on the first body signal and a frequency of a pulse based on the pulse wave signal are located in substantially the same frequency domain. In this case, it is determined that the pulse wave signal and the first body motion signal overlap.

In this way, a situation is observed in which a type of the body motion signal overlapping the pulse wave signal is different depending on exercise habits and an exercise capability even if the same exercise is carried out. Therefore, in the method in the past, it is likely that the pulse wave signal is suppressed or deleted together with the body motion signal (the first or second body motion signal) in signal processing. It is difficult to calculate an accurate pulse rate.

When the user is the person not having the athlete's heart, the overlap determining section 9207 determines overlap of the pulse wave signal and the body motion signal on the basis of a frequency of a pulse and a frequency of a body motion corresponding to the first body motion signal. On the other hand, when the user is the person having the athlete's heart, the overlap determining section 9207 determines overlap of the pulse wave signal and the body motion signal on the basis of the frequency of the pulse and a frequency of a body motion corresponding to the first body motion signal and a frequency of a body motion corresponding to the second body motion signal. When it is determined that the pulse wave signal and the body motion signal overlap, a pulse rate is calculated on the basis of the frequency of the body motion determined as overlapping. Consequently, even in a situation in which the pulse wave signal and the body motion signal overlap, it is possible to suppress the erroneous determination from being performed and calculate an accurate pulse rate. Therefore, in a user having any exercise habits, it is possible to appropriately count a pulse.

On the other hand, when the user wearing the measuring device 1 carries out exercise under a low temperature environment, since the surface temperature of the skin of the user in contact with the measuring device 1 is low, a detected pulse wave signal is small (weak) compared with the body motion signal.

In such a case, even if overlap of the pulse wave signal and the body motion signal is determined for the user not having the athlete's heart on the basis of a frequency of a pulse and a frequency of a body motion based on the first body motion signal, since the pulse wave signal mainly includes body motion noise components indicated by the body motion signal (mainly the first body motion signal), it is likely that the body motion noise components included in the pulse wave signal are specified as a pulse and it is erroneously determined that the pulse wave signal and the body motion signal overlap. In particular, immediately after an exercise start, a frequency of a body motion component indicated by the first body motion signal (e.g., a body motion component equivalent to one reciprocation of an arm swing of the user) and a frequency of an estimated pulse rate estimated on the basis of a pulse rate of the last pulse calculated on the basis of the pulse wave signal are extremely close to each other. Therefore, it is more likely that the body motion component is erroneously determined as a pulse.

On the other hand, when it is determined that the user is not the person having the athlete's heart, overlap of the pulse wave signal and the body motion signal based on the frequency of the pulse and the frequency of the body motion based on the first body motion signal is not determined. Consequently, it is possible to switch determination content when the user is the person having the athlete's heart and when the user is not the person having the athlete's heart. Therefore, it is possible to appropriately calculate a pulse rate according to a user.

### Second Embodiment

A second embodiment of the invention is explained.

A biological-information analyzing system according to this embodiment includes a detecting device that detects biological information and body motion information of a user and an analyzing device that analyzes the biological information and the body motion information received from the detecting device. The analyzing device executes the processing executed by the signal processing section 8 and the control section 9 to thereby realize a function same as the function of the biological-information measuring device 1. The biological-information analyzing system according to this embodiment and the biological-information measuring device 1 are different in this point. Note that, in the following explanation, portions same as or substantially same as the portions already explained are denoted by the same reference numerals and signs and explanation of the portions is omitted.

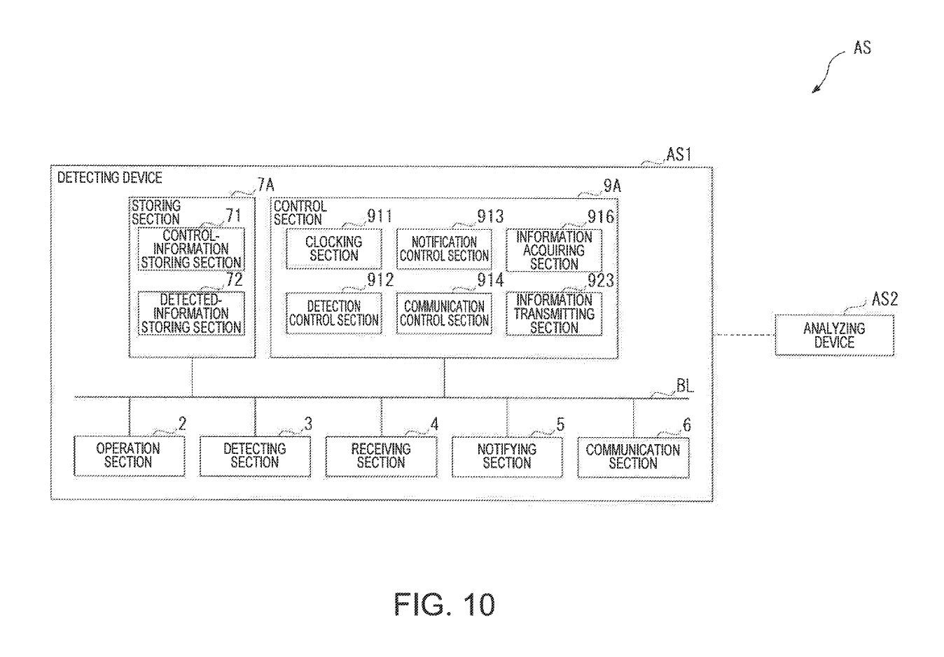

FIG. 10 is a block diagram showing the configuration of a detecting device AS1 configuring a biological-information analyzing system AS according to this embodiment.

The biological-information analyzing system AS according to this embodiment includes a detecting device AS1 and an analyzing device AS2 as shown in FIG. 10 and has a function same as the function of the measuring device 1.

### [Configuration of the Detecting Device]

The detecting device AS1 has a configuration same as the configuration of the measuring device 1 except that the detecting device AS1 includes a storing section 7A and a control section 9A instead of the storing section 7 and the control section 9 and does not include the signal processing section 8.

Among the sections, like the storing section 7, the storing section 7A includes the control-information storing section 71 and the detected-information storing section 72 but does not include the table storing section 73.

The control section 9A controls the operation of the detecting device AS1. The control section 9A includes the clocking section 911, the detection control section 912, the notification control section 913, the communication control section 914, the information acquiring section 916, and an information transmitting section 923. That is, the control section 9A has a configuration same as the configuration of the control section 9 except that the control section 9A does not include the analysis control section 915, the pace calculating section 917, the table selecting section 918, the pulse estimating section 919, the analyzing section 920, the update-condition determining section 921, and the table updating section 922 and, on the other hand, includes the information transmitting section 923.

Among the sections, as explained above, the information acquiring section 916 acquires various kinds of information input from the operation section 2, the detecting section 3, the receiving section 4, and the communication section 6. That is, the information acquiring section 916 acquires information (e.g., a pulse rate as an analysis result of biological information and body motion information) received from the analyzing device AS2 via the communication section 6. The information is notified by the notifying section 5, for example, under the control by the notification control section 913.

The information transmitting section 923 transmits biological information (a pulse wave signal) and body motion information (a body motion signal) detected by the detecting section 3 and stored in the detected-information storing section 72 to the analyzing device AS2 via the communication section 6.

### [Configuration of the Analyzing Device]

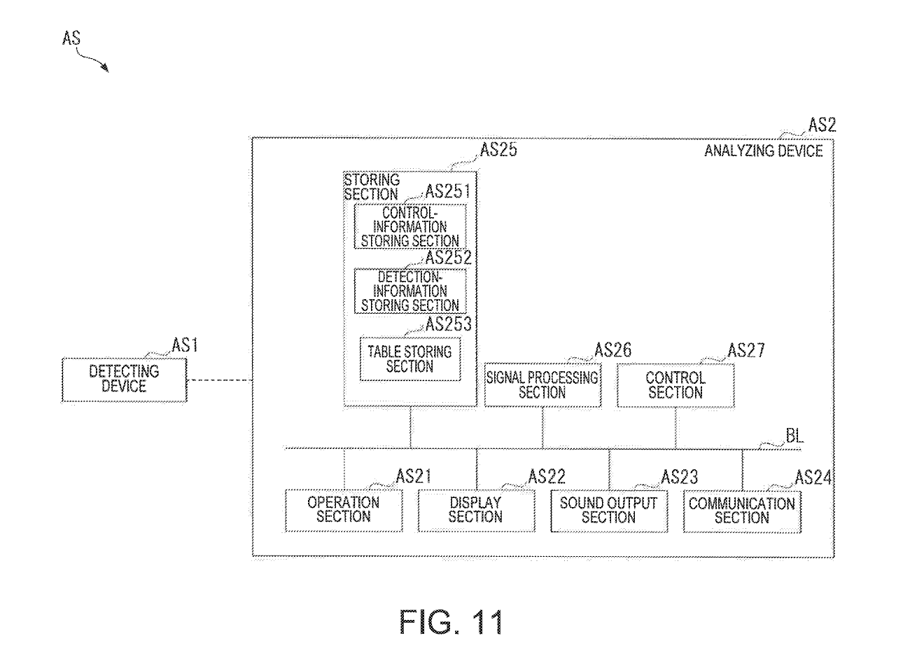

FIG. 11 is a block diagram showing the configuration of the analyzing device AS2 configuring the biological-information analyzing system AS.

The analyzing device AS2 analyzes biological information and body motion information received from the detecting device AS1 and transmits an analysis result to the detecting device AS1. The analyzing device AS2 can be configured by, for example, a PC (Personal Computer) or a smartphone (a multifunction cellular phone). The analyzing device AS2 includes, as shown in FIG. 11, an operation section AS21, a display section AS22, a sound output section AS23, a communication section AS24, a storing section AS25, a signal processing section AS26, and a control section AS27. That is, the analyzing device AS2 can also be considered a pulse-rate measuring device that analyzes biological information and body motion information detected by the detecting device AS1 and measures a pulse rate.

The operation section AS21 includes a keyboard and a pointing device and outputs an operation signal corresponding to input operation of the user to the control section AS27.

The display section AS22, the sound output section AS23, the communication section AS24, and the signal processing section AS26 respectively have configurations same as the configurations of the display section 51, the sound output section 52, the communication section 6, and the signal processing section 8.

Like the storing section 7, the storing section AS25 includes a control-information storing section AS251, a detected-information storing section AS252, and a table storing section AS253.

The control-information storing section AS251 has stored therein control information such as various programs (including an OS (Operating System)) and data necessary for the operation of the analyzing device AS2.

The detected-information storing section AS252 stores biological information and body motion information received from the detecting device AS1 via the communication section AS24 under the control by the control section AS27 explained below.

The table storing section AS253 has stored therein the table serving as analysis information.

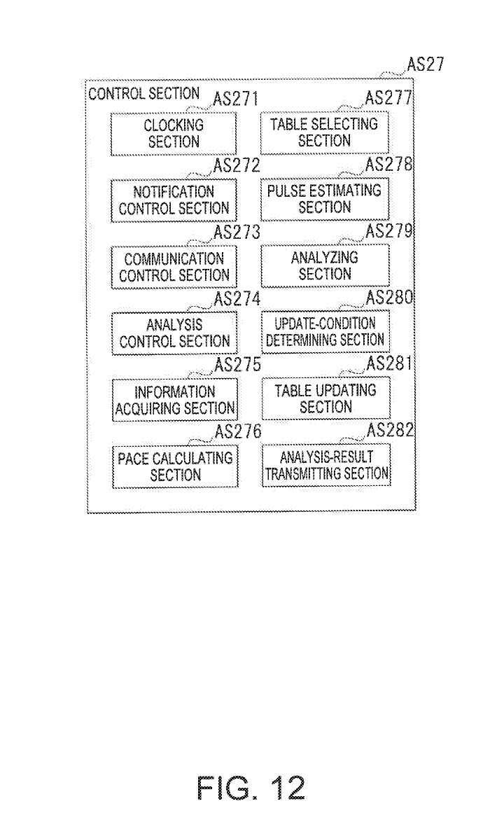

FIG. 12 is a block diagram showing the configuration of the control section AS27.

The control section AS27 includes an arithmetic processing circuit such as a CPU and controls the operation of the analyzing device AS2 autonomously or according to an operation signal input from the operation section AS21. The control section AS27 executes, for example, processing same as the processing executed by the control section 9 of the measuring device 1 and executes, on the basis of biological information and body motion information received from the detecting device AS1, selection of a table and analysis of the respective kinds of information (e.g., calculation of a pulse rate).

Therefore, the control section AS27 includes, as shown in FIG. 12, a clocking section AS271, a notification control section AS272, a communication control section AS273, an analysis control section AS274, an information acquiring section AS275, a pace calculating section AS276, a table selecting section AS277, a pulse estimating section AS278, an analyzing section AS279, an update-condition determining section AS280, a table updating section AS281, and an analysis-result transmitting section AS282.

Among the sections, the functional sections AS271 to AS281 respectively have functions same as the functions of the functional sections 911 and 913 to 922. For example, the notification control section AS272 causes the display section AS22 to display the setting screen SP. The information acquiring section AS275 acquires input content to the displayed setting screen SP. The communication control section AS273 controls the operation of the communication section AS24 that communicates with the detecting device AS1. The analysis control section AS274 controls the operation of the signal processing section AS26.

Further, the control section AS27 executes processing (first selection processing and second selection processing) for selecting the table on the basis of received biological information and body motion information mainly with the information acquiring section AS275, the pace calculating section AS276, the table selecting section AS277, the pulse estimating section AS278, the analyzing section AS279, the update-condition determining section AS280, and the table updating section AS281. Besides, the control section AS27 executes pulse rate measurement processing including the pulse rate calculation processing SD.

The analysis-result transmitting section AS282 transmits a pulse rate, which is an analysis result of the biological information and the body motion information and is a processing result of the pulse rate measurement processing, to the detecting device AS1 with the communication control section AS273 and the communication section AS24. Consequently, the pulse rate is displayed on the display section 51 configuring the notifying section 5 of the detecting device AS1.

### Effect of the Second Embodiment

With the biological-information analyzing system AS according to this embodiment explained above, it is possible to achieve an effect same as the effect of the biological-information measuring device 1 and can achieve an effect explained below.

The biological information and the body motion information detected by the detecting device AS1 are transmitted to the analyzing device AS2 and analyzed by the analyzing device AS2. Consequently, since processing having a relatively large processing load can be executed by the analyzing device AS2, it is possible to reduce the detecting device AS1 in size and reduce a processing load of the detecting device AS1.

### Modifications of the Embodiments

The invention is not limited to the embodiments. Modifications, improvements, and the like in a range in which the object of the invention can be achieved are included in the invention.

In the embodiments, the measuring device 1 and the analyzing device AS2 retain, as the analysis information, the table in which the pace and the estimated pulse rate are associated. However, the measuring device 1 and the analyzing device AS2 are not limited to this and may store, as the analysis information, a formula in which the pace and the estimated pulse rate are associated. The measuring device 1 and the analyzing device AS2 may retain analysis information for analyzing other biological information. For example, the measuring device 1 and the analyzing device AS2 may retain, as the analysis information, a table in which a pace and a respiration rate are associated or may retain a table in which the number of times of a swing of an arm or a foot per unit time and a pulse rate are associated. Further, the measuring device 1 and the analyzing device AS2 may retain a table in which the magnitude (power) of acceleration and a pulse rate are associated.

Further, the analysis information is a table used in calculation of a pulse rate based on a pulse wave signal, which is biological information. However, the analysis information is not limited to this and may be, for example, correction information of a signal detected as biological information. That is, the analysis information only has to be information used for analysis of biological information.

In the embodiments, examples of the analysis information include the table for ordinary people, the table for runners, and the table for athlete's heart. Any one of the tables can be selected on the setting screen SP. However, the analysis information is not limited to this. For example, instead of the tables, analysis information corresponding to an exercise capability and proficiency of exercise (e.g., a beginner, an intermediate level person, a high level person, an expert, and a master) of the user can be set. In this case, a configuration may be adopted in which at least two or more of these kinds of analysis information are presented to the user, analysis information corresponding to operation of the user is selected and set, and biological information is analyzed on the basis of the selected analysis information, whereby, for example, an estimated pulse rate is acquired.

The table may be selected and set on the basis of an exercise record of the user besides the operation of the user. For example, the analysis information such as the table may be selected on the basis of exercise achievement information including at least one of a cumulative time, cumulative intensity, and an exercise frequency of exercise carried out by the user.

In the embodiments, the exercise capability of the user is grasped on the basis of the resting pulse rate and the pulse rate corresponding to the exercise state. It is determined according to the exercise capability whether the user is the person having the athlete's heart. However, the grasp of the exercise capability is not limited to this. The exercise capability of the user may be grasped and determined on the basis of a cardiac output instead of or in addition to the resting pulse rate. Further, the exercise capability of the user may be grasped and determined on the basis of other information such as a muscle amount.

The analysis information is not always set according to whether the user is the person having the athlete's heart. Like the setting of the table for ordinary people or the table for runners, the analysis information corresponding to the exercise capability of the user only has to be set.

In the embodiments, it is determined on the basis of the user information input on the setting screen SP or the detected biological information and body motion information whether the user is the person having the athlete's heart. However, the determination is not limited to this. For example, the measuring device 1 and the analyzing device AS2 may receive, from the outside, information for determining whether the user is the person having the athlete's heart. The detecting device AS1 may transmit the information to the analyzing device AS2.

In the embodiments, the overlap determining section 9207 determines whether the difference between the frequency of the last pulse or the frequency of the pulse calculated from the estimated pulse rate and the frequency of the body motion is within the predetermined range to thereby determine whether the pulse wave signal and the body motion signal overlap. However, the determination is not limited to this. It may be determined according to other methods whether the pulse wave signal and the body motion signal overlap.

In the embodiments, when the SN-ratio determining section 9204 determines that the SN ratio of the pulse wave signal is lower than the predetermined value, the estimated pulse rate is acquired from the selected table. However, the acquisition of the estimated pulse rate is not limited to this. The estimated pulse rate may be acquired when signal intensity of the pulse wave signal is lower than a predetermined value. Further, the determination processing may be omitted.

In the embodiments, when the user is the person having the athlete's heart, it is determined whether the first body motion signal and the pulse wave signal overlap. However, the determination is not limited to this. Even if the user is not the person having the athlete's heart, it may be determined whether the body motion signal and the pulse wave signal overlap. That is, the determination processing for determining whether the user is the person having the athlete's heart can be omitted. On the other hand, the overlap determination for determining whether the second body motion signal and the pulse wave signal overlap may be executed only when the user is the person not having the athlete's heart.

In the embodiments, the frequency of the pulse is acquired as the pulse related information related to the periodicity of the pulse wave signal on the basis of the pulse wave signal or the power spectrum, which is the result of the frequency analysis of the pulsation signal obtained by removing the body motion noise components from the pulse wave signal. The value obtained by multiplying the frequency with 60 is set as the pulse rate. The frequency of the body motion is acquired as the body motion related information related to the periodicity of the body motion signal on the basis of the acceleration signal, which is the body motion signal indicating the body motion. However, the pulse related information and the body motion related information are not limited to this and may be other information. For example, the pulse related information and the body motion related information may be waveforms, cycles, and phases of the signals. In this case, a pulse rate may be calculated on the basis of a waveform of the pulse or may be calculated on the basis of a cycle of the pulse. When overlap of the pulse wave signal and the body motion signal is determined, a cycle and a phase of the pulse and a cycle and a phase of the body motion are compared. When appearance timing of the pulse and appearance timing of the body motion substantially coincide with each other (a difference between the appearance timing of the pulse and the appearance timing of the body motion is within a predetermined range), it may be determined that the pulse wave signal and the body motion signal coincide with each other.

In the embodiments, the measuring device 1 is configured as the wearable device worn on the wrist of the user. However, the measuring device 1 is not limited to this. That is, a wearing part of the measuring device 1 may be anywhere and may be, for example, a foot (more specifically, an ankle) or a chest. Depending on the wearing part, the first body motion signal does not have to be detected.

Further, in the embodiments, the body motion signal detected by the body-motion-information detecting section 32 includes the first body motion signal indicating the acceleration that changes according to the movement in one cycle of the reciprocating motion of the wearing part (e.g., the wrist or the foot) of the measuring device 1 and the detecting device AS1 (the arm swing of one reciprocation in the front-back direction during walking and during running) and the second body motion signal indicating the acceleration that changes according to the body motion in the half cycle of the reciprocating motion of the wearing part (one arm swing in the forward direction or the backward direction during walking and during running), that is, the pace of the user during walking and ruing running. However, the first body motion signal and the second body motion signal are not limited to this. Acceleration signals indicating body motions of the user in other directions may be set as the first body motion signal and the second body motion signal. For example, an acceleration change involved in a body motion in the up-down direction of the user may be set as the second body motion signal. A signal obtained by multiplying the second body motion signal with an integer equal to or larger than 2 may be set as the first body motion signal.

In the first embodiment, the measuring device 1 is configured to be usable alone. However, the invention is not limited to this. That is, the functions of the measuring device 1 and the functions of the detecting device AS1 and the analyzing device AS2 may be incorporated in an electronic device (e.g., a medical device).

### REFERENCE SIGNS LIST

1 biological-information measuring device (biological-information analyzing device)
2 operation section
31 biological-information detecting section
32 body-motion-information detecting section
51 display section
917 pace calculating section
9181 input-information determining section (user determining section)
9184 pulse determining section (user determining section)
9185 table setting section (information setting section)
920 analyzing section
923 information transmitting section
AS biological-information analyzing system
AS1 detecting device
AS2 analyzing device
AS277 table selecting section (user determining section, information setting section)
AS279 analyzing section
SP setting screen (screen)

1. A biological-information analyzing device comprising:
a biological-information detecting section configured to detect biological information of a user;
a user determining section configured to determine an exercise capability of the user;
an information setting section configured to set, when the user determining section determines that the exercise capability of the user satisfies a predetermined condition, analysis information corresponding to the exercise capability of the user; and
an analyzing section configured to analyze the biological information on the basis of the analysis information set by the information setting section.

2. The biological-information analyzing device according to claim 1, wherein the exercise capability is a capability concerning a cardiopulmonary function.

3. The biological-information analyzing device according to claim 2, wherein the user determining section determines the capability concerning the cardiopulmonary function on the basis of at least one of a resting pulse rate and a cardiac output.

4. The biological-information analyzing device according to claim 1, further comprising an operation section configured to receive input operation by the user, wherein
the user determining section determines the exercise capability of the user on the basis of content of the input operation.

5. The biological-information analyzing device according to claim 4, further comprising a display section configured to display a screen to which user information concerning the user is input, wherein
the user determining section determines the exercise capability of the user on the basis of the input user information.

6. The biological-information analyzing device according to claim 1, further comprising a body-motion-information detecting section configured to detect body motion information of the user, wherein
the user determining section determines the exercise capability of the user on the basis of an exercise state of the user based on the detected body motion information and biological information of the user in the exercise state.

7. The biological-information analyzing device according to claim 1, wherein
the biological information includes at least a pulse wave, and
the analyzing section calculates a pulse rate of the user on the basis of the pulse wave.

8. The biological-information analyzing device according to claim 7, further comprising a body-motion-information detecting section configured to detect body motion information of the user, wherein
the analysis information is a table in which an exercise state of the user based on the body motion information detected by the body-motion-information detecting section and a pulse rate are associated, and
the analyzing section calculates the pulse rate on the basis of the table.

9. The biological-information analyzing device according to claim 8, further comprising a pace calculating section configured to calculate a pace of the user on the basis of the body motion information, wherein
the table is a table in which a pace and a pulse rate are associated, and
the analyzing section acquires, from the table, a pulse rate corresponding to the pace of the user calculated by the pace calculating section and calculates the pulse rate of the user on the basis of the pulse rate and a pulse rate based on the biological information.

10. The biological-information analyzing device according to claim 1, further comprising a body-motion-information detecting section configured to detect a body motion signal of the user, wherein
the body-motion-information detecting section detects a biological signal for calculating the biological information, and
the user determining section determines the exercise capability on the basis of an overlapping state of frequencies of the biological signal and the body motion signal.

11. A biological-information analyzing system that analyzes biological information of a user, the biological-information analyzing system determining an exercise capability of the user, setting, when determining that the exercise capability of the user satisfies a predetermined condition, analysis information corresponding to the exercise capability of the user and, analyzing the biological information on the basis of the set analysis information.

12. A biological-information analyzing method for analyzing biological information of a user, the biological-information analyzing method comprising:
determining an exercise capability of the user;
setting, when determining that the exercise capability of the user satisfies a predetermined condition, analysis information corresponding to the exercise capability of the user; and
analyzing the biological information on the basis of the set analysis information.

13. A biological-information analyzing system comprising:
a detecting device configured to detect biological information of a user; and
an analyzing device configured to analyze the biological information detected by the detecting device, wherein
the detecting device includes:
a biological-information detecting section configured to detect the biological information; and
an information transmitting section configured to transmit the biological information detected by the biological-information detecting section, and
the analyzing device includes:
a user determining section configured to determine whether the user is a person having an athlete's heart;
an information setting section configured to set, when the user determining section determines that the user is the person having the athlete's heart, analysis information for the athlete's heart in analysis information used for the analysis of the biological information; and
an analyzing section configured to analyze, on the basis of the analysis information for the athlete's heart set by the information setting section, the biological information received from the detecting device.

14. A biological-information analyzing method for analyzing biological information of a user, the biological-information analyzing method comprising:
determining whether the user is a person having an athlete's heart;
setting, when it is determined that the user is the person having the athlete's heart, analysis information for the athlete's heart in analysis information used for the analysis of the biological information; and
analyzing the biological information on the basis of the set analysis information for the athlete's heart.
